(19) United States
(12) Patent Application Publication
(10) Pub. No.: US 2010/0183717 A1
(43) Pub. Date: Jul. 22, 2010

(54) CONTROLLED-RELEASE FORMULATIONS

(76) Inventors: Kristin Arnold, Morrisville, PA (US); Zhongshui Yu, Bridgewater, NJ (US); Siva Rama K. Nutalapati, Princeton, NJ (US)

Correspondence Address: CANTOR COLBURN, LLP, 20 Church Street, 22nd Floor, Hartford, CT, 06103, US

(21) Appl. No.: 12/687,966

(22) Filed: Jan. 15, 2010

Related U.S. Application Data
(60) Provisional application No. 61/145,136, filed on Jan. 16, 2009.
(60) Provisional application No. 61/145,807, filed on Jan. 20, 2009.

(51) Int. Cl. A61K 9/30; A61K 31/517; A61P 9/12; A61P 13/00; A61P 13/08; A61P 15/00

(52) U.S. Cl. 424/465; 514/266.24

(57) ABSTRACT

Disclosed herein are controlled-release formulations of a core comprising a core active agent (e.g., alfuzosin) and a wax excipient substantially coated with an extended-release coating.

### CROSS REFERENCE TO RELATED APPLICATION

This application claims the benefit of U.S. Provisional Application Ser. Nos. 61/145,136 filed Jan. 16, 2009 and 61/145,807 filed Jan. 20, 2009, which are hereby incorporated by reference in their entirety.

### BACKGROUND

Controlled-release dosage formulations, including sustained-release formulations, provide a variety of benefits to the patient such as reduction in the number of doses per day, increased convenience, reduced occurrences of missed doses, and the chance to achieve controlled blood levels of the active agent.

Alfuzosin, (R,S)—N-[3-[(4-amino-6,7-dimethoxy-2-quinazolinyl)methylamino]propyl]tetrahydro-2-furancarboxamide, is a selective antagonist of post-synaptic alpha1-adrenoreceptors, which are located in the prostate, bladder base, bladder neck, prostatic capsule, and prostatic urethra.

Alfuzosin hydrochloride is marketed in the United States under the brand name UROXATRAL® and elsewhere as XATRAL. The dosage form currently approved for marketing in the United States is a 10 mg extended release oral tablet (approved for marketing by the U.S. Food and Drug Administration (FDA) under NDA #021287 on Jun. 12, 2003). The UROXATRAL® 10 mg extended-release tablet is a round trilayer tablet comprising as inactive ingredients: colloidal silicon dioxide (NF), ethylcellulose (NF), hydrogenated castor oil (NF), hydroxypropyl methylcellulose (USP), magnesium stearate (NF), mannitol (USP), microcrystalline cellulose (NF), povidone (USP), and yellow ferric oxide (NF). The three-layer tablet has one white layer between two yellow layers and is debossed with X10.

The pharmacokinetics of UROXATRAL® have been evaluated in adult healthy male subjects. Under fed conditions following multiple dosing of 10 mg UROXATRAL®, Cₘₐₓ is 13.6±5.6 (s.d.) ng/mL with a Tₘₐₓ of 8 hrs and AUC₀₋₂₄ of 194±75 (s.d.) ng-hr/mL. These steady-state values are reported to be 1.2 to 1.6 fold higher than those observed after a single administration, corresponding to a Cₘₐₓ of 8.5-11.3 ng/mL. The extent of absorption of UROXATRAL® is also reported to be 50% lower under fasting conditions than under fed (non-fasted) conditions (UROXATRAL® prescribing information, Sanofi-Aventis, U.S., LLC, August 2007).

The recommended dosage is one 10 mg alfuzosin HCl extended-release tablet daily, to be taken immediately after the same meal each day. The UROXATRAL® extended-release tablets should not be chewed or crushed.

There remains a need for improved oral pharmaceutical formulations for the controlled release of active agents such as alfuzosin to allow for reduced incidents of administration, specifically single daily dose administrations. Also needed are dosage formulations having reduced or substantially no food effect such that a patient has the convenience of taking the dosage formulation with or without food.

### SUMMARY

In one embodiment, a controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material.

In another embodiment, a controlled-release formulation comprises a core comprising an alpha-1 receptor antagonist agent, (e.g., alfuzosin) or a pharmaceutically acceptable salt, solvate, hydrate, crystalline form, or non-crystalline form thereof, and a wax excipient; and an extended-release coating substantially surrounding the core comprising a release-retarding coating material; wherein the formulation exhibits reduced or substantially no food effect.

These and other embodiments, advantages and features of the present invention become clear when detailed description and examples are provided in subsequent sections.

### DETAILED DESCRIPTION

Disclosed herein are controlled-release formulations comprising a core comprising a core active agent and a wax excipient; and an extended-release coating substantially surrounding the core comprising a release-retarding coating material.

Also disclosed herein are formulations comprising a core comprising a core active agent and a wax excipient; and an extended-release coating substantially surrounding the core comprising a release-retarding coating material; wherein the formulation exhibits substantially no food effect. Furthermore, by choosing the appropriate core materials and coating materials, the resulting formulation possesses enough strength to resist rupture or significant damage to the dosage formulation that could result in a compromise of the release properties. It has been found that a core of a particular hardness (“strength”, e.g., about 10 to about 15 kilopascals (kPa)) provides sufficient support for the controlled-release coating to ensure the integrity of the coating when the formulation is ingested with food.

The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”). The endpoints of all ranges directed to the same component or property are inclusive and independently combinable.

An “active agent” means a compound, element, or mixture that when administered to a patient, alone or in combination with another compound, element, or mixture, confers, directly or indirectly, a physiological effect on the patient. The indirect physiological effect may occur via a metabolite or other indirect mechanism. When the active agent is a compound, then salts, solvates (including hydrates) of the free compound or salt, crystalline forms, non-crystalline forms, and any polymorphs of the compound are contemplated herein. Compounds may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In addition, compounds with carbon-carbon double bonds may occur in Z- and E-forms, with all isomeric forms of the compounds. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

“Pharmaceutically acceptable salts” includes derivatives of the active agent, wherein the active agent is modified by making acid or base addition salts thereof, and further refers to pharmaceutically acceptable solvates, including hydrates, crystalline forms, non-crystalline forms, and polymorphs of such salts. Examples of pharmaceutically acceptable salts include, but are not limited to, mineral or organic acid addition salts of basic residues such as amines; alkali or organic addition salts of acidic residues; and the like, and a combination comprising at least one of the foregoing salts. The pharmaceutically acceptable salts include salts and the quaternary ammonium salts of the active agent. For example, acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and a combination comprising at least one of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like; and a combination comprising at least one of the foregoing salts.

“Alfuzosin” means alfuzosin or a pharmaceutically acceptable alfuzosin salt, including any solvate, hydrate, crystalline form, and non-crystalline form thereof unless otherwise indicated. A specific alfuzosin salt is alfuzosin hydrochloride.

“Reference drug” means an alfuzosin product as described in U.S. Federal Food and Drug Administration's New Drug Application No. 021287 approved on Jun. 12, 2003 (10 mg) as provided in the U.S. Federal Food and Drug Administration's Orange Book, Approved Drug Products with Therapeutic Equivalence Evaluations. UROXATRAL® is an alfuzosin oral, extended-release tablet product available in 10 mg strength. UROXATRAL®, 10 mg is the “reference listed drug” under 21 CFR 314.94(a)(3)), i.e., the listed drug identified by FDA as the drug product upon which an applicant relies in seeking approval of its ANDA.

A “dosage form” or “dosage formulation” means a unit of administration of an active agent. Examples of dosage formulations include tablets, capsules, injections, suspensions, liquids, emulsions, creams, ointments, suppositories, inhalable formulations, transdermal formulations, and the like. “Form” and “formulation” are to be used interchangeably unless indicated otherwise.

By “oral dosage form” is meant to include a unit dosage form for oral administration. An oral dosage form may optionally comprise a plurality of subunits such as, for example, microcapsules or microtablets. Multiple subunits may be packaged for administration in a single dose.

By “subunit” is meant to include a composition, mixture, particle, pellet, and the like, that can provide an oral dosage form alone or when combined with other subunits.

“Bioavailability” means the extent or rate at which an active agent is absorbed into a living system or is made available at the site of physiological activity. For active agents that are intended to be absorbed into the bloodstream, bioavailability data for a given formulation may provide an estimate of the relative fraction of the administered dose that is absorbed into the systemic circulation. “Bioavailability” can be characterized by one or more pharmacokinetic parameters.

“Pharmacokinetic parameters” describe the in vivo characteristics of an active agent (or surrogate marker for the active agent) over time, such as plasma concentration (C), Cₘₐₓ, Cₙ, C₂₄, Tₘₐₓ, and AUC. “Cₘₐₓ” is the measured concentration of the active agent in the plasma at the point of maximum concentration. “Cₙ” is the measured concentration of an active agent in the plasma at about n hours after administration. “C₂₄” is the measured concentration of an active agent in the plasma at about 24 hours after administration. The term “Tₘₐₓ” refers to the time at which the measured concentration of an active agent in the plasma is the highest after administration of the active agent. “AUC” is the area under the curve of a graph of the measured concentration of an active agent (typically plasma concentration) vs. time, measured from one time point to another time point. For example AUC₀₋ₜ is the area under the curve of plasma concentration versus time from time 0 to time t. The AUC_{0-∞} or AUC_{0-INF} is the calculated area under the curve of plasma concentration versus time from time 0 to time infinity.

“Food” typically means a solid food or mixed solid/liquid food with sufficient bulk and fat content that it is not rapidly dissolved and absorbed in the stomach. In one embodiment, food means a meal, such as breakfast, lunch or dinner. The terms “taken with food”, “fed” and “non-fasted” are equivalent and are as given by FDA guidelines and criteria.

In one embodiment, with food means that the dosage form is administered to a patient between about 30 minutes prior to about 2 hours after eating a meal. In another embodiment, with food means that the dosage form is administered at substantially the same time as the eating the meal.

The terms “without food”, “fasted” and “an empty stomach” are equivalent and are as given by FDA guidelines and criteria. In one embodiment, fasted is means the condition wherein no food is consumed within 1 hour prior to administration of the dosage form or 2 hours after administration of the dosage form. In another embodiment, fasted means the condition wherein no food is consumed within 1 hour prior to administration of the dosage form to 2 hours after administration of the dosage form.

“Substantially no food effect” means that the pharmacokinetics are substantially the same for the oral administration of the formulation under fed conditions (“non-fasting”) when compared to administration under fasting conditions. For example, the comparison between Cₘₐₓ or AUC of a single administration of a formulation under fed conditions to a single administration of the same formulation under fasted conditions results in a percent ratio of Cₘₐₓ or AUC having a 90% confidence interval upper limit of less than or equal to 125% or a lower limit of greater than or equal to 80%. Such information can be based on logarithmic transformed data. Exemplary study considerations can be found in the Federal Drug Administration's (FDA) guidelines and criteria, including “Guidance for Industry, Food-Effect Bioavailability and Fed Bioequivalence Studies” available from the U.S. Department of Health and Human Services (DHHS), Food and Drug Administration (FDA), Center for Drug Evaluation and Research (CDER) December 2002, incorporated herein in its entirety.

In one embodiment, the extent of absorption of alfuzosin is not greater than 40% lower under fasting conditions than under fed (non-fasted) conditions, specifically not greater than 30%, more specifically not greater than 20%, and more specifically not greater than 10% when tested in a group of five or more healthy humans. The extent of absorption can be made based on log transformed data.

A dissolution profile is a plot of the cumulative amount of active agent released from a formulation as a function of time. A dissolution profile can be measured utilizing the Drug Release Test <724>, which incorporates standard test USP 26 (Test <711>). A profile is characterized by the test conditions selected such as, for example, apparatus type, shaft speed, temperature, volume, and pH of the dissolution medium. More than one dissolution profile may be measured. For example, a first dissolution profile can be measured at a pH level approximating that of the stomach, and a second dissolution profile can be measured at a pH level approximating that of one point in the intestine or several pH levels approximating multiple points in the intestine.

A highly acidic pH may be employed to simulate the stomach and a less acidic to basic pH may be employed to simulate the intestine. By the term “highly acidic pH” is meant a pH of about 1 to about 4. A pH of about 1.2, for example, can be used to simulate the pH of the stomach. By the term “less acidic to basic pH” is meant a pH of greater than about 4 to about 7.5, specifically about 6 to about 7.5. A pH of about 6 to about 7.5, specifically about 6.8, can be used to simulate the pH of the intestine.

By “immediate-release” is meant a conventional or non-modified release in which greater then or equal to about 75% of the active agent is released within two hours of administration, specifically within one hour of administration.

By “controlled-release” is meant a dosage form in which the release of the active agent is controlled or modified over a period of time. Controlled can mean, for example, extended-, sustained-, delayed- or pulsed-release at a particular time. Alternatively, controlled can mean that the release of the active agent is extended for longer than it would be in an immediate-release dosage form, e.g., at least over several hours.

Dosage formulations can have both immediate-release and controlled-release characteristics, for example, a combination of immediate-release pellets and controlled-release pellets; immediate-release coating and controlled-release core including a tablet core; and the like. The immediate-release portion of a combination dosage form may be referred to as a loading dose.

The core can be in the form of a particle, a pellet, a bead, a tablet, and the like, specifically as a tablet.

“Bioequivalence” means the absence of a significant difference in the rate and extent to which the active agent or surrogate marker for the active agent in pharmaceutical equivalents or pharmaceutical alternatives becomes available at the site of action when administered in an appropriately designed study.

In one embodiment, bioequivalence is any definition thereof as promulgated by the U.S. Food and Drug Administration or any successor agency thereof. In a specific embodiment, bioequivalence is determined according to the Federal Drug Administration's (FDA) guidelines and criteria, including “GUIDANCE FOR INDUSTRY BIOAVAILABILITY AND BIOEQUVALENCE STUDIES FOR ORALLY ADMINISTERED DRUG PRODUCTS—GENERAL CONSIDERATIONS” available from the U.S. Department of Health and Human Services (DHHS), Food and Drug Administration (FDA), Center for Drug Evaluation and Research (CDER) March 2003 Revision 1; and “GUIDANCE FOR INDUSTRY STATISTICAL APPROACHES TO ESTABLISHING BIOEQUIVALENCE” DHHS, FDA, CDER, January 2001, both of which are incorporated herein in their entirety.

In an embodiment, bioequivalence of a composition to a reference drug is determined by an in vivo pharmacokinetic study to determine a pharmacokinetic parameter for the active agent composition. Specifically, bioequivalence can be determined by an in vivo pharmacokinetic study comparing a pharmacokinetic parameter for the two compositions. A pharmacokinetic parameter for the active agent composition or the reference drug can be measured in a single or multiple dose bioequivalence study using a replicate or a nonreplicate design. For example, the pharmacokinetic parameters for active agent composition of the present invention and for a reference drug can be measured in a single dose pharmacokinetic study using a two-period, two-sequence crossover design. Alternately, a four-period, replicate design crossover study may also be used. Single doses of the test composition and reference drug are administered and blood or plasma levels of the active agent are measured over time. Pharmacokinetic parameters characterizing rate and extent of active agent absorption are evaluated statistically.

The area under the plasma concentration-time curve from time zero to the time of measurement of the last quantifiable concentration (AUC₀₋ₜ) and to infinity (AUC_{0-∞}), Cₘₐₓ, and Tₘₐₓ can be determined according to standard techniques. Statistical analysis of pharmacokinetic data is performed on logarithmic transformed data (e.g., AUC₀₋ₜ, AUC_{0-∞}, or Cₘₐₓ data) using analysis of variance (ANOVA).

Under U.S. FDA guidelines, two products (e.g., an inventive alfuzosin formulation and UROXATRAL®) or methods (e.g., dosing under non-fasted versus fasted conditions) are bioequivalent if the 90% Confidence Interval (CI) limits for a ratio of the geometric mean of logarithmic transformed AUC_{0-∞}, AUC₀₋ₜ, and Cₘₐₓ for the two products or two methods are about 0.80 to about 1.25.

In another embodiment, bioequivalence is determined according to the European Medicines Agency (EMEA) document “Note for Guidance on the Investigation of Bioavailability and Bioequivalence”, issued Jul. 26, 2001, available from EMEA.

To show bioequivalency between two compounds or administration conditions pursuant to Europe's EMEA guidelines, the 90% CI limits for a ratio of the geometric mean of logarithmic transformed AUC_{0-∞} and AUC₀₋ₜ for the two products or methods are about 0.80 to about 1.25. The 90% CI limits for a ratio of the geometric mean of logarithmic transformed Cₘₐₓ for the two products or methods can have a wider acceptance range when justified by safety and efficacy considerations. For example the acceptance range can be about 0.70 to about 1.43, specifically about 0.75 to about 1.33, and more specifically about 0.80 to about 1.25.

In one embodiment, in a given experiment, an active agent composition is considered to be bioequivalent to the reference drug if both the Test/Reference ratio for the geometric mean of logarithmic transformed AUC_{0-∞} AUC₀₋ₜ, or Cₘₐₓ ratio along with its corresponding lower and upper 90% CI limits are within a lower limit of about 0.80 and an upper limit of about 1.25. Thus, for direct comparison between an inventive active agent composition and a reference drug, the pharmacokinetic parameters for the active agent composition and the reference drug can be determined in side-by side in the same pharmacokinetic study.

In some embodiments a single dose bioequivalence study is performed under non-fasted or fasted conditions.

In other embodiments, the single dose bioequivalence study is conducted between the active agent composition and the reference listed drug using the strength specified by the FDA in APPROVED DRUG PRODUCTS WITH THERAPEUTIC EQUIVALENCE EVALUATIONS(ORANGE BOOK).

In some embodiments, an in vivo bioequivalence study is performed to compare all active agent compositions with corresponding strengths of the reference drug (e.g., 5 mg, 10 mg, or 20 mg of the active agent). In other embodiments, an in vivo bioequivalence study is performed only for the active agent composition of the present invention at the strength of the reference listed drug product (e.g., the highest approved strength) and at the other lower or higher strengths, the inventive compositions meet a reference product dissolution test.

In one embodiment, a controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, wherein the core is monolithic and not prepared as having distinct layers. In a further embodiment, the controlled-release formulation is a unitary tablet formulation comprising a monolithic core and an extended-release coating substantially surrounding the core.

In one embodiment, the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, wherein the formulation administered as 10 mg dosage strength is bioequivalent to a reference drug product according to New Drug Application No. 021287, (UROXATRAL®, 10 mg) when tested in a group of five or more healthy humans in the non-fasting state.

In another embodiment, the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, wherein the formulation administered as 10 mg dosage strength exhibits a ratio of a geometric mean of logarithmic transformed AUC_{0-∞} of the formulation to a geometric mean of logarithmic transformed AUC_{0-∞} of reference drug New Drug Application No. 021287, (UROXATRAL®, 10 mg) of about 0.80 to about 1.25 when tested in a group of five or more healthy humans in the non-fasting state.

In another embodiment, the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, wherein the formulation administered as 10 mg dosage strength exhibits a ratio of a geometric mean of logarithmic transformed AUC₀₋ₜ of the formulation to a geometric mean of logarithmic transformed AUC₀₋ₜ of reference drug New Drug Application No. 021287, (UROXATRAL®, 10 mg) of about 0.80 to about 1.25 when tested in a group of five or more healthy humans in the non-fasting state.

In another embodiment, the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, wherein the formulation administered as 10 mg dosage strength exhibits a ratio of a geometric mean of logarithmic transformed Cₘₐₓ of the formulation to a geometric mean of logarithmic transformed Cₘₐₓ of reference drug New Drug Application No. 021287, (UROXATRAL®, 10 mg) of about 0.70 to about 1.43 when tested in a group of five or more healthy humans in the non-fasting state.

In another embodiment, the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, wherein the formulation administered as 10 mg dosage strength exhibits a ratio of a geometric mean of logarithmic transformed Cₘₐₓ of the formulation to a geometric mean of logarithmic transformed Cₘₐₓ of reference drug New Drug Application No. 021287, (UROXATRAL®, 10 mg) of about 0.80 to about 1.25 when tested in a group of five or more healthy humans in the non-fasting state.

In one embodiment, the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, wherein the formulation (administered as 2×10 mg dosage forms) is bioequivalent to a reference drug product according to New Drug Application No. 021287, (UROXATRAL®, 10 mg) when tested in a group of five or more healthy humans in the non-fasting state.

In another embodiment, the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, wherein the formulation (administered as 2×10 mg dosage forms) exhibits a ratio of a geometric mean of logarithmic transformed AUC_{0-∞} of the formulation to a geometric mean of logarithmic transformed AUC_{0-∞} of reference drug New Drug Application No. 021287, (UROXATRAL®, 10 mg) of about 0.80 to about 1.25 when tested in a group of five or more healthy humans in the non-fasting state.

In another embodiment, the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, wherein the formulation (administered as 2×10 mg dosage forms) exhibits a ratio of a geometric mean of logarithmic transformed AUC₀₋ₜ of the formulation to a geometric mean of logarithmic transformed AUC₀₋ₜ of reference drug New Drug Application No. 021287, (UROXATRAL®, 10 mg) of about 0.80 to about 1.25 when tested in a group of five or more healthy humans in the non-fasting state.

In another embodiment, the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, wherein the formulation (administered as 2×10 mg dosage forms) exhibits a ratio of a geometric mean of logarithmic transformed Cₘₐₓ of the formulation to a geometric mean of logarithmic transformed Cₘₐₓ of reference drug New Drug Application No. 021287, (UROXATRAL®, 10 mg) of about 0.70 to about 1.43 when tested in a group of five or more healthy humans in the non-fasting state.

In another embodiment, the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, wherein the formulation (administered as 2×10 mg dosage forms) exhibits a ratio of a geometric mean of logarithmic transformed Cₘₐₓ of the formulation to a geometric mean of logarithmic transformed Cₘₐₓ of reference drug New Drug Application No. 021287, (UROXATRAL®, 10 mg)) of about 0.80 to about 1.25 when tested in a group of five or more healthy humans in the non-fasting state.

In one embodiment, the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material; wherein the formulation exhibits a reduced food effect.

In still another embodiment, the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, wherein the formulation exhibits a ratio of a geometric mean of logarithmic transformed AUC_{0-∞} of the formulation administered in a non-fasted state to a geometric mean of logarithmic transformed AUC_{0-∞} of the formulation administered in a fasted state of about 0.80 to about 1.25 when tested in a group of five or more healthy humans.

In one embodiment, the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, wherein the formulation exhibits a ratio of a geometric mean of logarithmic transformed AUC₀₋ₜ of the formulation administered in a non-fasted state to a geometric mean of logarithmic transformed AUC₀₋ₜ of the formulation administered in a fasted state of about 0.80 to about 1.25 when tested in a group of five or more healthy humans.

In an embodiment, the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, wherein the formulation exhibits a reduced food effect such that an area under the alfuzosin plasma concentration curve from time 0 to infinity (AUC_{0-∞}) of the formulation administered in a fasted state is no more than 25% lower than when the formulation is administered in a non-fasted state, specifically about 1 to about 20%, more specifically about 5 to about 15% and yet more specifically about 10 to about 12% lower than when the formulation is administered in a non-fasted state, when tested in a group of five or more healthy humans.

In an embodiment, the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, wherein the formulation exhibits a reduced food effect such that an area under the alfuzosin plasma concentration curve from time 0 to time t (AUC₀₋ₜ) of the formulation administered in a fasted state is no more than 25% lower than when the formulation is administered in a non-fasted state, specifically about 1 to about 20%, more specifically about 5 to about 15% and yet more specifically about 10 to about 12% lower than when the formulation is administered in a non-fasted state when tested in a group of five or more healthy humans.

In an embodiment, the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, wherein the formulation exhibits a reduced food effect such that a maximum alfuzosin plasma concentration (Cₘₐₓ) of the formulation administered in a fasted state is no more than 35% lower than when the formulation is administered in a non-fasted state, specifically about 1 to about 30%, more specifically about 5 to about 25% and yet more specifically about 10 to about 20% lower than when the formulation is administered in a non-fasted state when tested in a group of five or more healthy humans in the non-fasting state.

In one embodiment, the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, wherein the formulation administered as 10 mg dosage strength exhibits a ratio of a geometric mean of logarithmic transformed AUC₀₋ₜ of the formulation administered in a fasted state to a geometric mean of logarithmic transformed AUC₀₋ₜ of reference drug New Drug Application No. 021287, (UROXATRAL®, 10 mg)) administered in a non-fasted state of about 0.80 to about 1.25 when tested in a group of five or more healthy humans.

In one embodiment, the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, wherein the formulation administered as 10 mg dosage strength in the fasted state is bioequivalent to a reference drug product according to New Drug Application No. 021287, (UROXATRAL®, 10 mg) administered in a non-fasted state when tested in a group of five or more healthy humans.

In one embodiment, the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, wherein the formulation administered as 10 mg dosage strength exhibits a ratio of a geometric mean of logarithmic transformed AUC_{0-∞} of the formulation administered in a fasted state to a geometric mean of logarithmic transformed AUC_{0-∞} of reference drug New Drug Application No. 021287, (UROXATRAL®, 10 mg)) administered in a non-fasted state of about 0.80 to about 1.25 when tested in a group of five or more healthy humans.

In one embodiment, the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, wherein the formulation exhibits a ratio of a geometric mean of logarithmic transformed Cₘₐₓ of the formulation administered as 10 mg dosage strength administered in a fasted state to a geometric mean of logarithmic transformed Cₘₐₓ of reference drug New Drug Application No. 021287, (UROXATRAL®, 10 mg)) administered in a non-fasted state of about 0.80 to about 1.25 when tested in a group of five or more healthy humans.

In one embodiment, the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, wherein the formulation administered as 10 mg dosage strength exhibits a ratio of a geometric mean of logarithmic transformed Cₘₐₓ of the formulation administered in a fasted state to a geometric mean of logarithmic transformed Cₘₐₓ of reference drug New Drug Application No. 021287, (UROXATRAL®, 10 mg)) administered in a non-fasted state of about 0.70 to about 1.43 when tested in a group of five or more healthy humans.

The formulations disclosed herein comprise a core comprising an active agent, a wax excipient, and optionally additional core excipients.

The wax excipient for use in the core can be a solid wax at ambient temperature, such as a solid, hydrophobic material (i.e., non-water soluble) or solid hydrophilic material (e.g., polyethylene glycols are water soluble), but specifically a solid, hydrophobic material.

Exemplary wax excipients include wax and wax-like excipients, for example, carnauba wax (from the palm tree Copernicia Cerifera), vegetable wax, fruit wax, microcrystalline wax (“petroleum wax”), bees wax (white or bleached, and yellow), hydrocarbon wax, paraffin wax, cetyl esters wax, non-ionic emulsifying wax, anionic emulsifying wax, candelilla wax, or a combination comprising at least one of the foregoing waxes. Other suitable wax excipients include, for example, fatty alcohols (such as lauryl, myristyl, stearyl, cetyl or specifically cetostearyl alcohol), hydrogenated vegetable oil, hydrogenated castor oil, fatty acids such as stearic acid, fatty acid esters including fatty acid glycerides (mono-, di-, and tri-glycerides), polyethylene glycol (PEG) having a molecular weight of greater than about 3000 number average molecular weight, Mₙ, (e.g., PEG 3350, PEG 4000, PEG 4600, PEG 6000, and PEG 8000), or a combination comprising at least one of the foregoing wax excipients. Any combination of wax excipients is also contemplated.

The melting point of the wax excipient is a temperature above ambient temperature, specifically about 30 to about 150° C., more specifically about 75 to about 100° C., and yet more specifically about 75 to about 90° C.

The amount of wax excipient present in the core can be determined based on the particular wax or wax combination chosen and the targeted release profile desired for the resulting formulation. Exemplary amounts of a wax excipient include about 5 to about 70 wt. % based on the total weight of the core excluding the extended-release coating, specifically about 10 to about 60 wt. %, more specifically about 15 to about 55 wt. %, yet more specifically about 20 to about 50 wt. %, still yet more specifically about 25 to about 45 wt. %, and more specifically about 30 to about 40 based on the total weight of the core excluding the extended-release coating.

In another embodiment, the core may comprise an active agent that is an alpha-1 receptor antagonist agent such as alfuzosin hydrochloride. Exemplary amounts of active agent in the core include about 1.0 to about 20 wt. % based on the total weight of the core excluding the extended-release coating, specifically about 2.0 to about 15 wt. %, and more specifically about 3.0 to about 10 wt. %.

In one embodiment, the active agent is alfuzosin hydrochloride. The formulation can contain about 1 mg to about 20 mg of alfuzosin hydrochloride, specifically about 5 mg to about 15 mg, and more specifically about 10 mg to about 12 mg per unit. In one embodiment, the formulation is a tablet containing about 10 to about 20 mg of alfuzosin hydrochloride per tablet, specifically about 10 to about 12 mg per tablet.

The core optionally further contains an additional release-retarding material. Additional release-retarding materials include, for example an acrylic polymer, an alkylcellulose including substituted alkylcellulose, shellac, zein, polyvinylpyrrolidine including crosslinked polyvinylpyrrolidinone, a vinyl acetate copolymer, a polyethylene oxide, a polyvinyl alcohol, and a combination comprising at least one of the foregoing materials.

Suitable acrylic polymers for use as an additional release-retarding material include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, or a combination comprising at least one of the foregoing polymers. The acrylic polymer may comprise methacrylate copolymers described in NF XXIV as fully polymerized copolymers of acrylic and methacrylic acid esters with a low content of quaternary ammonium groups.

Suitable alkylcelluloses and substituted alkyl celluloses include, for example, methyl cellulose, ethylcellulose, hydroxy or carboxy substituted alkyl celluloses (e.g., hydroxyl propylcellulose, crosslinked hydroxypropylcellulose, carboxymethylcellulose, crosslinked sodium carboxymethylcellulose), hydroxy substituted alkyl-alkyl celluloses (e.g., hydroxypropylmethylcellulose), or a combination comprising at least one of the foregoing alkyl celluloses.

The additional release-retarding material is present in the core in an amount of 0.1 to about 65 wt. % based on the total weight of the core, specifically about 1 to about 50 wt. %, more specifically about 2 to about 35 wt. %, yet more specifically about 2.5 to about 20 wt. %, and yet more specifically about 5 to about 10 wt. %. Besides the additional release-retarding material, the additional core excipients optionally include binders, fillers, disintegrants, lubricants, glidants, and the like.

In one embodiment, the core is free of a hydrophilic polymer such as hydroxymethyl cellulose, hydroxyethyl cellulose, hydroxypropyl methylcellulose, hydroxypropyl cellulose, and the like.

Exemplary fillers include, for example, microcrystalline cellulose, colloidal silicon dioxide, a saccharide (e.g., lactose, sucrose, and the like); an inorganic filler (e.g., calcium carbonate, calcium sulfate, dibasic calcium phosphate, sodium chloride, and the like); a sugar alcohol (e.g., sorbitol, lactitol, mannitol, maltitol, xylitol, and the like); and a combination comprising at least one of the foregoing materials. Exemplary combinations include mannitol-microcrystalline cellulose or lactose-microcrystalline cellulose.

Exemplary amounts of filler include about 1.0 to about 60 wt. % based on the total weight of the core, specifically about 5.0 to about 50 wt. %, more specifically about 10 to about 45 wt. %, still more specifically about 20 to about 40 wt. %, and yet more specifically about 25 to about 35 wt. %.

The optional disintegrant is used to facilitate the breakdown of the core in a fluid environment, specifically aqueous environments. The choice and amount of disintegrant is tailored to ensure the desired dissolution profile of the formulation or to provide the desired controlled-release in vivo. Exemplary disintegrants include a material that possesses the ability to swell or expand upon exposure to a fluid environment, especially an aqueous environment. Exemplary disintegrants include hydroxyl substituted alkyl celluloses (e.g., hydroxypropyl cellulose), starch, pregelatinized starch (e.g., Starch 1500® available from Colorcon); cross-linked sodium carboxymethylcellulose (e.g., “croscarmellose sodium”, i.e., Ac-Di-Sol® available from FMC BioPolymer of Philadelphia, Pa.); crosslinked homopolymer of N-vinyl-2-pyrrolidone (e.g., “crospovidone”, e.g., Polyplasdone® XL, Polyplasdone® XL-10, and Polyplasdone® INF-10 available from International Specialty Products, Wayne N.J.); modified starches, such as sodium carboxymethyl starch, sodium starch glycolate (e.g., Primogel®), and the like; alginates; or a combination comprising at least one of the foregoing disintegrants.

The amount of disintegrant used depends upon the disintegrant or disintegrant combination chosen and the targeted release profile of the resulting formulation. Exemplary amounts include about 0 to about 10 wt. % based on the total weight of the core, specifically about 0.5 to about 7.0 wt. %, and yet more specifically about 0.1 to about 5.0 wt. %.

Exemplary lubricants include stearates (e.g., calcium stearate, magnesium stearate, and zinc stearate), sodium stearyl fumarate, mineral oil, talc, or a combination comprising at least one of the foregoing. Glidants include, for example, silicon dioxide (e.g., fumed or colloidal). It is recognized that certain materials can function both as a glidant and a lubricant.

The lubricant or glidant is used in amounts of about 0.1 to about 15 wt. % of the total weight of the core; specifically about 0.5 to about 5 wt. %; and yet more specifically about 0.75 to about 3 wt. %.

The cores are prepared by processes known in the art, including granulation (dry or wet) and compression, spheronization, melt extrusion, hot fusion, and the like.

Once the core is formed, it is coated with an extended-release coating. The extended-release coating that substantially surrounds the core comprises a release-retarding coating material and optional other components, such as plasticizers, pore formers, and the like.

“Substantially surrounding the core” means the coating covers more than 90% of the surface area of the core, specifically more than 95%, more specifically more than 98%, and yet more specifically more than 99%.

The extended-release coating is present in the formulation at about 0.1 to about 30 wt. % based on the total weight of the core and extended-release coating, specifically about 1.0 to about 25 wt. %, more specifically about 2.5 to about 20 wt. %, yet more specifically about 3.5 to about 15 wt. %, still yet more specifically about 5.0 to about 12 wt. %, and yet more specifically about 5.5 to about 10 wt. %.

The extended-release coating is provided on the core using known coating processes such as simple or complex coacervation, interfacial polymerization, liquid drying, thermal and ionic gelation, spray drying, spray chilling, fluidized bed coating, pan coating, electrostatic deposition, compression coating, dry polymer powder coating, and the like.

The release-retarding coating material is, for example, in the form of a film coating comprising a dispersion of a hydrophobic polymer. Solvents used for application of the controlled-release coating include pharmaceutically acceptable solvents, such as water, methanol, ethanol, methylene chloride, and a combination comprising at least one of the foregoing solvents.

The extended-release profile of the active agent (either in vivo or in vitro) can be altered, for example, by using more than one release-retarding coating material, varying the thickness of the release-retarding coating material, changing the particular release-retarding coating material used, altering the relative amounts of release-retarding coating material, use of a plasticizer, altering the manner in which the plasticizer is added (e.g., when the extended-release coating is derived from an aqueous dispersion of hydrophobic polymer), by varying the amount of plasticizer relative to release-retarding coating material, by the inclusion of an additional coating excipient, by altering the method of manufacture, and the like.

Exemplary release-retarding coating materials include film-forming polymers such as an alkylcellulose including methylcellulose or ethylcellulose, a hydroxyalkylcellulose such as hydroxymethyl cellulose, hydroxyethylcellulose, hydroxypropylcellulose and hydroxybutylcellulose, a hydroxyalkyl alkylcellulose such as hydroxyethyl methylcellulose and hydroxypropyl methylcellulose, a carboxyalkylcellulose such as carboxymethylcellulose, an alkali metal salt of carboxyalkylcelluloses such as sodium carboxymethylcellulose, a carboxyalkyl alkylcellulose such as carboxymethyl ethylcellulose, a carboxyalkylcellulose ester, a starch, a pectin such as sodium carboxymethylamylopectine, a chitin derivate such as chitosan, a polysaccharide such as alginic acid, alkali metal and ammonium salts thereof, a carrageenan, a galactomannan, traganth, agar-agar, gum arabicum, guar gum and xanthan gum, a polyacrylic acid and the salts thereof, a polymethacrylic acid and the salts thereof, a methacrylate copolymer, a polyvinylalcohol, a polyvinylpyrrolidone, a copolymer of polyvinylpyrrolidone with vinyl acetate, a polyalkylene oxide such as polyethylene oxide and polypropylene oxide and a copolymer of ethylene oxide and propylene oxide, or a combination comprising at least one of the foregoing materials.

The controlled-release coating optionally comprises a plasticizer, an additional film-former, a pore former, or a combination comprising at least one of the foregoing materials. In one embodiment, the release-retarding coating material comprises a plasticizer or a combination of plasticizers in an amount to substantially prevent cracking of the coating during typical use and handling. Exemplary plasticizers include fatty acids and medium-chain triglycerides (e.g., fractionated coconut oil), polyethylene glycol, dibutyl sebacate, diethyl phthalate, triethyl citrate, tributyl citrate, triacetin, acetylated monoglycerides, phthalate esters, castor oil, and the like, or a combination comprising one or more of the foregoing plasticizers. Exemplary amounts of plasticizer to substantially prevent cracking of the coating can be about 0.1 to about 40% wt based on the weight of the coating after drying, specifically about 1.0 to about 30, more specifically about 2.0 to about 25% wt.

The formulations optionally further comprises a non-functional coating. By “functional coating” is meant to include a coating that modifies the release properties of the total formulation, for example, a controlled-release coating that provides sustained-release. By “non-functional coating” is meant to include a coating that does not significantly modify the release properties of the total formulation, for example, a cosmetic coating or an interlayer coating used to separate a functional coating from other components of the formulation. A non-functional coating can have some impact on the release of the active agent due to the initial dissolution, hydration, perforation of the coating, and the like, but would not be considered to be a significant deviation from the non-coated composition.

A pore forming material is optionally be added to the controlled-release coating to promote release of the active agent from the core. The pore forming material is organic or inorganic; it is a material that can be dissolved, extracted or leached from the coating in the environment of use; or it can have a pH-dependent solubility property; and the like. Exemplary pore forming materials include hydrophilic polymers such as a hydroxy alkyl-alkyl cellulose (e.g., hydroxypropylmethyl cellulose, and the like), a hydroxyl alkyl cellulose (e.g., hydroxypropylcellulose, and the like), or a povidone; a saccharide (e.g., lactose, and the like); a metal stearate; an inorganic salt (e.g., dibasic calcium phosphate, sodium chloride, and the like); a polyethylene glycol (e.g., polyethylene glycol (PEG) 1450, and the like); a sugar alcohol (e.g., sorbitol, mannitol, and the like); an alkali alkyl sulfate (e.g., sodium lauryl sulfate); a polyoxyethylene sorbitan fatty acid ester (e.g., polysorbate); methyacrylate copolymers (e.g., EUDRAGIT® RL); or a combination comprising at least one of the foregoing pore forming materials.

A specific release-retarding coating material includes ethyl cellulose and optionally in combination with hydroxypropymethyl cellulose.

In one embodiment, the ratio of ethyl cellulose to hydroxypropylmethyl cellulose in the coating is about 90:10, specifically about 60:40, and yet more specifically about 50:50.

In another embodiment, the ratio of ethyl cellulose to hydroxypropylmethyl cellulose in the coating is about 60:40 to about 50:50, specifically about 57:43 to about 52:48, and more specifically about 55:45 to about 53:47.

In yet another embodiment, a controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, where in the extended-release coating is present in the formulation at about 0.1 to about 30 wt. % based on the total weight of the core and extended-release coating, specifically about 4.0 to about 25 wt. %, more specifically about 6.0 to about 20 wt. %, yet more specifically about 8.0 to about 15 wt. %, and still yet more specifically about 10.0 to about 12 wt. %; and the extended-release coating comprises a ratio of ethyl cellulose to hydroxypropylmethyl cellulose in the extended-release coating of about 60:40 to about 50:50, specifically about 57:43 to about 52:48, and more specifically about 55:45 to about 53:47.

In one embodiment, the formulation is free of an enteric coating.

In one embodiment, the controlled-release core releases the core active agent over a period of about 6 hours to about 24 hours, specifically about 12 hours or about 24 hours.

In one embodiment, the formulation comprises a controlled-release portion in the form of a coated core and an immediate-release portion disposed on at least a portion of the controlled-release core. The formulation is prepared, for example, as a bilayer tablet, a coated tablet, a compression-coated tablet, or any other suitable form. In one embodiment, the immediate-release portion is in the form of a coating substantially surrounding the coated core applied, for example, using spray coating, compression coating, or other suitable technique. The active agent present in the controlled-release portion and the immediate-release portion can be the same or different.

In one embodiment, the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, wherein the formulation exhibits a dissolution profile in a sequential in vitro dissolution study having a sequence containing dissolution media of 500 ml 0.1N HCl for the first two hours, 250 ml 0.350M Sodium Acetate pH 4.5 buffer for hours 3 and 4, and 250 ml of 0.125M Tribasic sodium phosphate pH 6.8 buffer for hours 6 to 23, the study is performed at 37° C.±0.5° C. according to USP 28 <711> test method 2 (paddle), 100 rpm paddle speed and a Sandwich sinker, wherein about 0 to about 30 wt. % of the total amount of active agent is released 1 hour after combining the formulation with the dissolution media.

In another embodiment, the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, wherein the formulation exhibits a dissolution profile in a sequential in vitro dissolution study having a sequence containing dissolution media of 500 ml 0.1N HCl for the first two hours, 250 ml 0.350M Sodium Acetate pH 4.5 buffer for hours 3 and 4, and 250 ml of 0.125M Tribasic sodium phosphate pH 6.8 buffer for hours 6 to 23, the study is performed at 37° C.±0.5° C. according to USP 28 <711> test method 2 (paddle), 100 rpm paddle speed and a Sandwich sinker, wherein about 10 to about 40 wt. % of the total amount of active agent is released 2 hours after combining the formulation with the dissolution media.

In yet another embodiment, the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, wherein the formulation exhibits a dissolution profile in a sequential in vitro dissolution study having a sequence containing dissolution media of 500 ml 0.1N HCl for the first two hours, 250 ml 0.350M Sodium Acetate pH 4.5 buffer for hours 3 and 4, and 250 ml of 0.125M Tribasic sodium phosphate pH 6.8 buffer for hours 6 to 23, the study is performed at 37° C.±0.5° C. according to USP 28 <711> test method 2 (paddle), 100 rpm paddle speed and a Sandwich sinker, wherein about 15 to about 50 wt. % of the total amount of active agent is released 3 hour after combining the formulation with the dissolution media.

In still another embodiment, the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, wherein the formulation exhibits a dissolution profile in a sequential in vitro dissolution study having a sequence containing dissolution media of 500 ml 0.1N HCl for the first two hours, 250 ml 0.350M Sodium Acetate pH 4.5 buffer for hours 3 and 4, and 250 ml of 0.125M Tribasic sodium phosphate pH 6.8 buffer for hours 6 to 23, the study is performed at 37° C.±0.5° C. according to USP 28 <711> test method 2 (paddle), 100 rpm paddle speed and a Sandwich sinker, wherein about 30 to about 65 wt. % of the total amount of active agent is released 6 hours after combining the formulation with the dissolution media.

In another embodiment, the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, wherein the formulation exhibits a dissolution profile in a sequential in vitro dissolution study having a sequence containing dissolution media of 500 ml 0.1N HCl for the first two hours, 250 ml 0.350M Sodium Acetate pH 4.5 buffer for hours 3 and 4, and 250 ml of 0.125M Tribasic sodium phosphate pH 6.8 buffer for hours 6 to 23, the study is performed at 37° C.±0.5° C. according to USP 28 <711> test method 2 (paddle), 100 rpm paddle speed and a Sandwich sinker, wherein about 50 to about 85 wt. % of the total amount of active agent is released 12 hours after combining the formulation with the dissolution media.

In another embodiment, the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material, wherein the formulation exhibits a dissolution profile in a sequential in vitro dissolution study having a sequence containing dissolution media of 500 ml 0.1N HCl for the first two hours, 250 ml 0.350M Sodium Acetate pH 4.5 buffer for hours 3 and 4, and 250 ml of 0.125M Tribasic sodium phosphate pH 6.8 buffer for hours 6 to 23, the study is performed at 37° C.±0.5° C. according to USP 28 <711> test method 2 (paddle), 100 rpm paddle speed and a Sandwich sinker, wherein about 65 to about 100 wt. % of the total amount of active agent is released 23 hours after combining the formulation with the dissolution media.

Also included herein are pharmaceutical kits which comprise one or more containers containing a controlled-release formulation, wherein the formulation comprises a core comprising an active agent and a wax excipient; and an extended-release coating substantially surrounding the core comprising a release-retarding coating material. The kits may further comprise one or more conventional pharmaceutical kit components, such as, for example, one or more containers to aid in facilitating compliance with a particular dosage regimen; one or more carriers; printed instructions, either as inserts or as labels, indicating quantities of the components to be administered, or guidelines for administration. Exemplary kits can be in the form of bubble or blister pack cards, optionally arranged in a desired order for a particular dosing regimen. Suitable blister packs that can be arranged in a variety of configurations to accommodate a particular dosing regimen are well known in the art or easily ascertained by one of ordinary skill in the art.

In one embodiment, a method of treating a patient comprises administering a controlled release alfuzosin formulation to a patient in need thereof, wherein the controlled-release formulation comprises a core comprising alfuzosin or a pharmaceutically acceptable salt, solvate, hydrate, crystalline form, or non-crystalline form thereof, and a wax excipient; and an extended-release coating substantially surrounding the core comprising a release-retarding coating material. The alfuzosin compositions are useful in treating or preventing conditions such as signs and symptoms of benign prostatic hyperplasia, urinary retention (including acute urinary retention), treating urinary problems associated with multiple sclerosis, chronic prostatitis, lower urinary tract symptoms, female sexual dysfunction, premature ejaculation, and primary dysmenorrheal, and for managing blood pressure or delaying onset of male ejaculation.

In one embodiment, a controlled-release formulation comprises a core comprising alfuzosin or a pharmaceutically acceptable salt, solvate, hydrate, crystalline form, or non-crystalline form thereof, about 40 to about 65 wt. % of a wax excipient based on the total weight of the core, and about 25 to about 50 wt. % of a filler based on the total weight of the core; and about 2.5 to about 15 wt. % of an extended-release coating based on the total weight of the formulation, where the extended-release coating substantially surrounds the core and comprises a release-retarding coating material. Within this embodiment, the wax is carnauba wax and the filler is mannitol. Further within this embodiment, the extended-release coating comprises ethyl cellulose and hydroxypropyl methylcellulose in a weight ratio of about 60:40 to about 50:50.

In one embodiment, a controlled-release formulation comprises a core comprising alfuzosin or a pharmaceutically acceptable salt, solvate, hydrate, crystalline form, or non-crystalline form thereof, about 45 to about 60 wt. % of a wax excipient based on the total weight of the core, and about 30 to about 45 wt. % of a filler based on the total weight of the core; and about 3.0 to about 12 wt. % of an extended-release coating based on the total weight of the formulation, where the extended-release coating substantially surrounds the core and comprises a release-retarding coating material. Within this embodiment, the wax is carnauba wax and the filler is mannitol. Further within this embodiment, the extended-release coating comprises ethyl cellulose and hydroxypropyl methylcellulose in a weight ratio of about 60:40 to about 50:50.

In one embodiment, a controlled-release formulation comprises a core comprising alfuzosin or a pharmaceutically acceptable salt, solvate, hydrate, crystalline form, or non-crystalline form thereof, about 40 to about 65 wt. % of a wax excipient based on the total weight of the core, about 25 to about 50 wt. % of a filler based on the total weight of the core, and about 1 to about 5 wt. % of an additional release-retarding material based on the total weight of the core; and about 2.5 to about 15 wt. % of an extended-release coating based on the total weight of the formulation, where the extended-release coating substantially surrounds the core and comprises a release-retarding coating material. Within this embodiment, the wax is carnauba wax, the additional release-retarding material is ethyl cellulose, and the filler is mannitol. Further within this embodiment, the extended-release coating comprises ethyl cellulose and hydroxypropyl methylcellulose in a weight ratio of about 60:40 to about 50:50.

In one embodiment, a controlled-release formulation comprises a core comprising alfuzosin or a pharmaceutically acceptable salt, solvate, hydrate, crystalline form, or non-crystalline form thereof, about 45 to about 60 wt. % of a wax excipient based on the total weight of the core, about 30 to about 45 wt. % of a filler based on the total weight of the core, and about 2 to about 2.5 wt. % of an additional release-retarding material based on the total weight of the core; and about 3 to about 12 wt. % of an extended-release coating based on the total weight of the formulation, where the extended-release coating substantially surrounds the core and comprises a release-retarding coating material. Within this embodiment, the wax is carnauba wax, the additional release-retarding material is ethyl cellulose, and the filler is mannitol. Further within this embodiment, the extended-release coating comprises ethyl cellulose and hydroxypropyl methylcellulose in a weight ratio of about 60:40 to about 50:50.

### EXAMPLES

### Example 1

### Alfuzosin Hydrochloride Extended-Release Tablets, 10 mg Tablet Cores

Extended-release alfuzosin hydrochloride tablet cores are prepared having the components listed in Table 1 below.

TABLE 1

| | Weight (mg/tablet) | | |
|---|---|---|---|
| Component | 1A | 1B | 1C |
| Alfuzosin HCl | 10 | 10 | 10 |
| Carnauba Wax | 120 | 90 | 100 |
| Mannitol | 62 | 92 | 82 |
| Ethyl cellulose (T10) | 5.0 | 5.0 | 5.0 |
| Denatured alcohol* | — | — | — |
| Colloidal Silicon dioxide | 2.0 | 2.0 | 2.0 |
| (CABOSIL) | | | |
| Magnesium Stearate | 1.0 | 1.0 | 1.0 |
| Total core tablet weight | 200 | 200 | 200 |

*Does not appear in final product

The tablet cores are prepared by mixing denatured alcohol with ethyl cellulose. Alfuzosin hydrochloride, carnauba wax, and mannitol are added to a mixer/granulator and mixed. The solution of ethyl cellulose is added to the dry mixture and granulated. The resulting granules are dried and milled. The milled granules are charged to a Gemco Blender to which screened silicon dioxide is added and mixed. Screened magnesium stearate is then added and mixed to form a blend. The resulting blend is then compressed into tablet cores to a hardness of about 9 to about 15 kp, specifically about 12 kp.

### Example 2

### Alfuzosin Hydrochloride Extended-Release Tablets

The tablet cores of Example 1 are then coated with an extended-release coating solution to achieve a targeted weight gain to form coated, extended-release alfuzosin hydrochloride tablets. The components of the coatings are listed in Table 2 below.

TABLE 2

| | 2A | 2B | 2C | 2D | 2E | 2F | 2G | 2H | 2I |
|---|---|---|---|---|---|---|---|---|---|
| Component | Weight (mg/tablet) | | | | | | | | |
| Tablet Cores | 200 | 200 | 200 | 200 | 200 | 200 | 200 | 200 | 200 |
| | (1B) | (1C) | (1A) | (1A) | (1B) | (1C) | (1A) | (1A) | (1A) |
| Surelease | 12.72 | 12.72 | 16.96 | 25.44 | 25.44 | 25.44 | 50.88 | 84.80 | 101.76 |
| 25% solids | (3.18 | (3.18 | (4.24 | (6.36 | (6.36 | (6.36 | (12.72 | (21.20 | (25.44 |
| (E-7-19010) | solids) | solids) | solids) | solids) | solids) | solids) | solids) | solids) | solids) |
| [Ethylcellulose- | | | | | | | | | |
| based | | | | | | | | | |
| coating] | | | | | | | | | |
| Opadry clear | 2.82 | 2.82 | 3.76 | 5.64 | 5.64 | 5.64 | 11.28 | 18.80 | 22.56 |
| (YS-3-7011) | | | | | | | | | |
| [Hypromellose- | | | | | | | | | |
| based | | | | | | | | | |
| coating] | | | | | | | | | |
| Purified | — | — | — | — | — | — | — | — | — |
| water* | | | | | | | | | |
| Coating | 3 | 3 | 4 | 6 | 6 | 6 | 12 | 20 | 24 |
| weight gain | | | | | | | | | |
| (%) | | | | | | | | | |
| Total tablet | 206 | 206 | 208 | 212 | 212 | 212 | 224 | 240 | 248 |
| weight | | | | | | | | | |

*Does not appear in final product

In a generalized coating process, Opadry clear is dissolved in water and added to a container containing Surelease with mixing to form a coating solution. The coating solution is applied to the wax tablet cores using a tablet spray coater. The coated tablets are dried in an oven at 60° C. for twenty-four hours.

### Example 3

### Dissolution

A sequential in vitro dissolution study was conducted on several coated tablets of Example 2. The sequence was 500 ml 0.1N HCl+250 ml 0.350M Sodium Acetate+250 ml of 0.125M Tribasic sodium phosphate. The test method protocol followed is USP 26, 711, at 37° C.±0.5° C. and a paddle speed of 100 rotations per minute (rpm) and a Sandwich sinker was used. The results of the dissolution analyses are summarized in Table 3.

TABLE 3

| | | | Example 2 | |
|---|---|---|---|---|
| | | Example 2 | Formulation | |
| | Time | Formulation 2G | 2H | |
| | (hr) | 12% wt gain | 20% wt gain | UROXATRAL ® |
| Medium | % drug dissolved | | | |
| — | 0 | 0 | 0 | 0 |
| 0.1N HCl | 1 | 17 | 6 | 14 |
| 0.1N HCl | 2 | 32 | 16 | 20 |
| pH4.5 buffer | 3 | 40 | 24 | 24 |
| pH4.5 buffer | 4 | 48 | 31 | 28 |
| pH6.8 buffer | 6 | 57 | 40 | 37 |
| pH6.8 buffer | 8 | 64 | 48 | 45 |
| pH6.8 buffer | 10 | 71 | 54 | 48 |
| pH6.8 buffer | 12 | 75 | 59 | 51 |
| pH6.8 buffer | 16 | 82 | 69 | 59 |
| pH6.8 buffer | 23 | 90 | 80 | 72 |

### Example 4

### Relative Bioavailability Under Non-Fasting Conditions of the Extended-Release Coated Tablet Formulation 2G of Example 2 (60% Wax 12% Coat) Versus UROXATRAL® (10 mg); and Food Effect Evaluation with Formulation 2G of Example 2, Fasting Versus Non-Fasting

The study is an open-label, single dose, randomized, three-period, three-treatment, fed relative bioavailability study of a 10 mg alfuzosin HCl tablet of formulation 2G Example 2 (60% wax 12% coat) compared to reference listed drug alfuzosin HCl extended-release tablet (10 mg, UROXATRAL®, Sanofi-Aventis) when administered under fed conditions (high fat breakfast) to healthy adult male volunteers. A food-effect evaluation is also performed with an additional 10 mg alfuzosin HCl tablet of formulation 2G Example 2 administered in a fasted state to the same group of volunteers. The study is performed on 44 subjects. Each subject receives a single 10 mg dose of alfuzosin HCl on three separate occasions in a randomly assigned sequence, separated by fourteen day washout periods. During each study period, all subjects receive either a single dose of formulation 2G under fasted conditions, a single dose of formulation 2G under fed conditions (high fat breakfast), or a single dose of UROXATRAL® under fed conditions depending upon their randomization scheme following a minimum overnight fast of at least ten hours. Blood samples are drawn from each subject for drug content analysis at time zero (predose) and after dose administration every ½ hour for the first eight hours, then at hours 9, 10, 12, 16, 20, 24, 36, 48, and 72. Alfuzosin plasma concentrations in the blood samples are measured using a validated bioanalytical method.

The alfuzosin concentration-time data are used to calculate the following pharmacokinetic parameters: AUC₀₋ₜ, AUC_{0-∞}, Cₘₐₓ, Tₘₐₓ, kₑ, and t_{1/2}. The pharmacokinetic parameters are evaluated statistically by an analysis of variance (ANOVA) appropriate for the experimental design of the study. Analyses for AUC₀₋ₜ, AUC_{0-∞}, and Cₘₐₓ are performed on ln-transformed data. Ninety percent confidence intervals (90% CI) for the ratio of fed test (formulation 2G administered in the fed state) versus fed reference (UROXATRAL® administered in the fed state) and 90% CI for the ratio of fasted test (formulation 2G administered in the fasted state) versus fed test (formulation 2G administered in the fed state) are calculated for AUC₀₋ₜ, AUC_{0-∞}, Cₘₐₓ, and Tₘₐₓ consistent with the two one-sided tests approach. The results are provided in Tables 4-7 below.

TABLE 4
Formulation 2G, Non-Fasting versus UROXATRAL ®, Non-fasting; N = 44

| | | | | |
|---|---|---|---|---|
| | | | | 90% |
| | | | | Confidence |
| | | Reference | % | Interval (Lower Limit, |
| PK variable | Formulation 2G | (UROXATRAL ®) | Ratio | Upper Limit) |

Ln-transformed data
Geometric Mean

| | | | | |
|---|---|---|---|---|
| Cₘₐₓ (ng/ml) | 11747.53 | 14051.68 | 83.60 | (74.84, 93.39) |
| AUC₀₋ₜ (ng- | 131564.26 | 175860.30 | 74.81 | (68.51, 81.69) |
| hr/ml) | | | | |
| AUC_{0-INF} (ng- | 149526.57 | 191651.41 | 78.02 | (71.69, 84.91) |
| hr/ml) | | | | |

Non-transformed data
least squares mean

| | | | | |
|---|---|---|---|---|
| Cₘₐₓ (ng/ml) | 13111.73 | 15646.84 | 83.80 | (72.74, 94.86) |
| AUC₀₋ₜ (ng- | 145332.73 | 191293.80 | 75.97 | (67.77, 84.17) |
| hr/ml) | | | | |
| AUC_{0-INF} (ng- | 162158.13 | 205338.15 | 78.97 | (71.21, 86.74) |
| hr/ml) | | | | |
| Tₘₐₓ | 4.01 | 6.41 | 62.57 | (52.75, 72.39) |
| kₑ | 0.0762 | 0.0887 | 86.00 | (76.7, 95.3) |
| t_{1/2} | 10.41 | 8.82 | 117.93 | (103.39, 132.47) |

TABLE 5
Formulation 2G, Non-Fasting versus UROXATRAL ®, Non-fasting; N = 43
(Excluded one subject due to emesis)

| | | | | |
|---|---|---|---|---|
| | | | | 90% Confidence |
| | | Reference | % | Interval (Lower Limit, |
| PK variable | Formulation 2G | (UROXATRAL ®) | Ratio | Upper Limit) |

Ln-transformed data
Geometric Mean

| | | | | |
|---|---|---|---|---|
| Cₘₐₓ (ng/ml) | 11569.87 | 14109.12 | 82.00 | (73.4, 91.61) |
| AUC₀₋ₜ (ng- | 129384.83 | 175914.98 | 73.55 | (67.41, 80.25) |
| hr/ml) | | | | |
| AUC_{0-INF} (ng- | 147442.23 | 191825.63 | 76.86 | (70.65, 83.62) |
| hr/ml) | | | | |

Non-transformed data
least squares mean

| | | | | |
|---|---|---|---|---|
| Cₘₐₓ (ng/ml) | 12899.93 | 15722.94 | 82.05 | (71, 93.09) |
| AUC₀₋ₜ (ng- | 142500.15 | 191517.33 | 74.41 | (66.28, 82.53) |
| hr/ml) | | | | |
| AUC_{0-INF} (ng- | 159543.27 | 205647.39 | 77.58 | (69.86, 85.3) |
| hr/ml) | | | | |
| Tₘₐₓ | 4.02 | 6.43 | 62.55 | (52.52, 72.58) |
| kₑ | 0.0764 | 0.0886 | 86.28 | (76.76, 95.8) |
| t_{1/2} | 10.42 | 8.83 | 117.94 | (103.07, 132.81) |

TABLE 6
Formulation 2G, Fasting versus Formulation 2G, Non-fasting; N = 44

| | | | | |
|---|---|---|---|---|
| | | | | 90% Confidence |
| | | | | Interval |
| | Formulation 2G | Formulation 2G | | (Lower Limit, |
| PK variable | Fasting | Non-fasting | % Ratio | Upper Limit) |

Ln-transformed data
Geometric Mean

| | | | | |
|---|---|---|---|---|
| Cₘₐₓ (ng/ml) | 9231.25 | 11747.53 | 78.58 | (70.35, 87.78) |
| AUC₀₋ₜ (ng-hr/ml) | 115747.06 | 131564.26 | 87.98 | (80.57, 96.07) |
| AUC_{0-INF} (ng-hr/ml) | 131127.81 | 149526.57 | 87.70 | (80.58, 95.44) |

Non-transformed data
least squares mean

| | | | | |
|---|---|---|---|---|
| Cₘₐₓ (ng/ml) | 10175.87 | 13111.73 | 77.61 | (64.41, 90.8) |
| AUC₀₋ₜ (ng-hr/ml) | 126756.68 | 145332.73 | 87.22 | (76.42, 98.01) |
| AUC_{0-INF} (ng-hr/ml) | 142653.22 | 162158.13 | 87.97 | (78.14, 97.8) |
| Tₘₐₓ | 4.51 | 4.01 | 112.51 | (96.81, 128.21) |
| kₑ | 0.08 | 0.08 | 102.16 | (91.35, 112.97) |
| t_{1/2} | 10.45 | 10.41 | 100.41 | (88.08, 112.73) |

TABLE 7
Formulation 2G, Fasting versus Formulation 2G, Non-fasting; N = 43 (Excluded one
subject due to emesis)

| | | | | |
|---|---|---|---|---|
| | | | | 90% Confidence |
| | | | | Interval |
| | Formulation 2G | Formulation 2G | | (Lower Limit, |
| PK variable | Fasting | Non-fasting | % Ratio | Upper Limit) |

Ln-transformed data
Geometric Mean

| | | | | |
|---|---|---|---|---|
| Cₘₐₓ (ng/ml) | 9156.96 | 11569.87 | 79.14 | (70.84, 88.42) |
| AUC₀₋ₜ (ng-hr/ml) | 114038.90 | 129384.83 | 88.14 | (80.78, 96.16) |
| AUC_{0-INF} (ng-hr/ml) | 129427.11 | 147442.23 | 87.78 | (80.69, 95.5) |

Non-transformed data
least squares mean

| | | | | |
|---|---|---|---|---|
| Cₘₐₓ (ng/ml) | 10101.44 | 12899.93 | 78.31 | (64.84, 91.77) |
| AUC₀₋ₜ (ng-hr/ml) | 124434.02 | 142500.15 | 87.32 | (76.4, 98.24) |
| AUC_{0-INF} (ng-hr/ml) | 140473.33 | 159543.27 | 88.05 | (78.1, 97.99) |
| Tₘₐₓ | 4.53 | 4.02 | 112.76 | (96.72, 128.8) |
| kₑ | 0.08 | 0.08 | 101.85 | (90.82, 112.89) |
| t_{1/2} | 10.47 | 10.42 | 100.49 | (87.89, 113.1) |

As shown by the results, a significant reduction of food effect is observed with formulation 2G when compared with UROXATRAL®. The extent of alfuzosin absorption is approximately 50% lower when UROXATRAL® is administered under fasted conditions as compared to administration of UROXATRAL® under non-fasting conditions (See, UROXATRAL® prescribing information, Sanofi-Aventis, U.S., LLC, August 2007 illustrating mean (SEM) alfuzosin plasma concentration-time profiles after a single-dose administration of UROXATRAL® Extended-release 10 mg tablets to healthy middle-aged male volunteers (N=8)). As shown in Tables 6-7, the alfuzosin plasma concentration is only about 20% lower when formulation 2G is administered under fasted conditions as compared to non-fasting conditions.

### Example 5

### Relative Bioavailability Under Non-Fasting Conditions of the Extended-Release Coated Tablet Formulation 2H of Example 2 (60% Wax 20% Coat) Versus UROXATRAL® (10 mg); and Food Effect Evaluation with Formulation 2H of Example 2, Fasting Versus Non-Fasting

A similar study to Example 4 is performed on 46 subjects. The study is an open-label, single dose, randomized, three-period, three-treatment, fed relative bioavailability study of a 10 mg alfuzosin HCl tablet of formulation 2H Example 2 (60% wax 20% coat) compared to reference listed drug alfuzosin HCl extended-release tablet (10 mg, UROXATRAL®, Sanofi-Aventis) when administered under fed conditions (high fat breakfast) to healthy adult male volunteers. A food-effect evaluation is also performed with an additional 10 mg alfuzosin HCl tablet of formulation 2H Example 2 administered in a fasted state to the same group of volunteers. The results are provided in Tables 8 and 9 below.

TABLE 8
Formulation 2H, Non-Fasting versus UROXATRAL ®, Non-fasting; N = 46

| | | | | |
|---|---|---|---|---|
| | | | | 90% Confidence |
| | | | | Interval |
| | | Reference | % | (Lower Limit, |
| PK variable | Formulation 2H | (UROXATRAL ®) | Ratio | Upper Limit) |

Ln-transformed data
Geometric Mean

| | | | | |
|---|---|---|---|---|
| Cₘₐₓ (ng/ml) | 6263.83 | 12953.80 | 48.36 | (42.98, 54.4) |
| AUC₀₋ₜ (ng-hr/ml) | 78347.76 | 168000.48 | 46.64 | (41.81, 52.02) |
| AUC_{0-INF} (ng- | 104725.21 | 182604.87 | 57.35 | (50.77, 64.78) |
| hr/ml) | | | | |

Non-transformed data
least squares mean

| | | | | |
|---|---|---|---|---|
| Cₘₐₓ (ng/ml) | 6923.73 | 13825.21 | 50.08 | (42.21, 57.95) |
| AUC₀₋ₜ (ng- | 89026.87 | 186410.60 | 47.76 | (40.31, 55.21) |
| hr/ml) | | | | |
| AUC_{0-INF} (ng- | 116740.34 | 199865.86 | 58.41 | (49.07, 67.75) |
| hr/ml) | | | | |
| Tₘₐₓ | 5.12 | 7.14 | 71.82 | (59, 84.64) |
| kₑ | 0.0706 | 0.0953 | 74.14 | (63.09, 85.18) |
| t_{1/2} | 14.64 | 7.92 | 184.97 | (134.37, 235.57) |

TABLE 9
Formulation 2H, Fasting versus Formulation 2H, Non-fasting; N = 46

| | | | | |
|---|---|---|---|---|
| | | | | 90% Confidence |
| | | | | Interval |
| | Formulation 2H | Formulation 2H | | (Lower Limit, |
| PK variable | Fasting | Non-fasting | % Ratio | Upper Limit) |

Ln-transformed data
Geometric Mean

| | | | | |
|---|---|---|---|---|
| Cₘₐₓ (ng/ml) | 4967.80 | 6263.83 | 79.31 | (70.5, 89.22) |
| AUC₀₋ₜ (ng-hr/ml) | 68061.65 | 78347.76 | 86.87 | (77.88, 96.9) |
| AUC_{0-INF} (ng-hr/ml) | 92950.87 | 104725.21 | 88.76 | (78.57, 100.26) |

Non-transformed data
least squares mean

| | | | | |
|---|---|---|---|---|
| Cₘₐₓ (ng/ml) | 5589.32 | 6923.73 | 80.73 | (65, 96.45) |
| AUC₀₋ₜ (ng-hr/ml) | 76235.54 | 89026.87 | 85.63 | (70.03, 101.23) |
| AUC_{0-INF} (ng-hr/ml) | 105278.55 | 116740.34 | 90.18 | (74.2, 106.17) |
| Tₘₐₓ | 6.05 | 5.12 | 118.03 | (100.18, 135.88) |
| kₑ | 0.07 | 0.07 | 96.52 | (81.63, 111.42) |
| t_{1/2} | 14.20 | 14.64 | 96.96 | (69.6, 124.31) |

As shown by the results, a significant reduction of food effect is observed with formulation 2H when compared with UROXATRAL®. As shown in Table 9, the alfuzosin plasma concentration is only about 20% lower when formulation 2H is administered under fasted conditions as compared to non-fasting conditions.

Also shown by the results in Table 8, formulation 2H would exhibit the same Cₘₐₓ and AUC when dosing 20 mg versus 10 mg of UROXATRAL®.

### Example 6

### Relative Bioavailability Under Non-Fasting Conditions of the Extended-Release Coated Tablet Formulation 2C of Example 2 (60% Wax 4% Coat) Versus UROXATRAL® (10 mg); Relative Bioavailability of the Extended-Release Coated Tablet Formulation 2C of Example 2 Under Fasting Conditions Versus UROXATRAL® (10 mg) Under Non-Fasting Conditions; and Food Effect Evaluation with Formulation 2C of Example 2, Fasting Versus Non-Fasting

The study is an open-label, single dose, randomized, three-period, three-treatment, fed/fasted bioequivalence study of a 10 mg alfuzosin HCl tablet of formulation 2C Example 2 (60% wax 4% coat) compared to reference listed drug alfuzosin HCl extended-release tablet (10 mg, UROXATRAL®, Sanofi-Aventis) when administered under fed conditions (high fat breakfast) to healthy adult male volunteers. A food-effect evaluation is also performed with an additional 10 mg alfuzosin HCl tablet of formulation 2C Example 2 administered in a fasted state to the same group of volunteers. The study is performed on 57 subjects. Each subject receives a single 10 mg dose of alfuzosin HCl, in either a fasted or fed state, on three separate occasions in a randomly assigned sequence, separated by fourteen day washout periods. During each study period, all subjects receive either a single dose of formulation 2C under fasted conditions, a single dose of formulation 2C under fed conditions (high fat breakfast), or a single dose of UROXATRAL® under fed conditions depending upon their randomization scheme following a minimum overnight fast of at least ten hours. For the fed treatment arms, the dose will be administered within five minutes of completing a standard, high-fat breakfast, and the subjects will have thirty minutes in which to complete the entire breakfast. Blood samples are drawn from each subject for drug content analysis at time zero (predose) and after dose administration every ½ hour for the first eight hours, then at hours 9, 10, 12, 16, 20, 24, 36, and 48. Alfuzosin plasma concentrations in the blood samples are measured using a validated bioanalytical method.

The alfuzosin concentration-time data are used to calculate the following pharmacokinetic parameters: AUC₀₋ₜ, AUC_{0-∞}, Cₘₐₓ, Tₘₐₓ, kₑ, and t_{1/2}. The pharmacokinetic parameters are evaluated statistically by an analysis of variance (ANOVA) appropriate for the experimental design of the study. Analyses for AUC₀₋ₜ, AUC_{0-∞}, and Cₘₐₓ are performed on ln-transformed data. Ninety percent confidence intervals (90% CI) for the ratio of fed test (formulation 2C administered in the fed state) versus fed reference (UROXATRAL® administered in the fed state); 90% CI for the ratio of fasted test (formulation 2C administered in the fasted state) versus fed reference (UROXATRAL® administered in the fed state); and 90% CI for the ratio of fasted test (formulation 2C administered in the fasted state) versus fed test (formulation 2C administered in the fed state) are calculated for AUC₀₋ₜ, AUC_{0-∞}, Cₘₐₓ, and Tₘₐₓ consistent with the two one-sided tests approach. The results are provided in Tables 10-12 below.

TABLE 10
Formulation 2C, Non-Fasting versus UROXATRAL ®, Non-fasting; N = 57

| | | | | |
|---|---|---|---|---|
| | | | | 90% Confidence |
| | | Reference | % | Interval (Lower Limit, |
| PK variable | Formulation 2C | (UROXATRAL ®) | Ratio | Upper Limit) |

Ln-transformed data
Geometric Mean

| | | | | |
|---|---|---|---|---|
| Cₘₐₓ (ng/ml) | 9749.23 | 13730.48 | 71.00 | (64.5, 78.17) |
| AUC₀₋ₜ (ng-hr/ml) | 114042.17 | 188228.84 | 60.59 | (55.76, 65.83) |
| AUC_{0-INF} (ng- | 127826.23 | 203288.57 | 62.88 | (57.86, 68.34) |
| hr/ml) | | | | |

Non-transformed data
least squares mean

| | | | | |
|---|---|---|---|---|
| Cₘₐₓ (ng/ml) | 10672.40 | 14852.34 | 71.86 | (63.57, 80.14) |
| AUC₀₋ₜ (ng- | 126368.34 | 204849.66 | 61.69 | (55.68, 67.7) |
| hr/ml) | | | | |
| AUC_{0-INF} (ng- | 140244.83 | 219505.75 | 63.89 | (57.83, 69.96) |
| hr/ml) | | | | |
| Tₘₐₓ | 2.91 | 6.60 | 44.11 | (33.89, 54.33) |
| kₑ | 0.0812 | 0.0846 | 95.97 | (83.11, 108.82) |
| t_{1/2} | 10.51 | 9.12 | 115.19 | (102.79, 127.59) |

TABLE 11
Formulation 2C, Fasting versus UROXATRAL ®, Non-fasting; N = 57

| | | | | |
|---|---|---|---|---|
| | | | | 90% Confidence |
| | | Reference | | Interval (Lower Limit, |
| PK variable | Formulation 2C | (UROXATRAL ®) | % Ratio | Upper Limit) |

Ln-transformed data
Geometric Mean

| | | | | |
|---|---|---|---|---|
| Cₘₐₓ (ng/ml) | 9184.69 | 13730.48 | 66.89 | (60.76, 73.64) |
| AUC₀₋ₜ (ng-hr/ml) | 112740.66 | 188228.84 | 59.90 | (55.13, 65.08) |
| AUC_{0-INF} (ng- | 130448.36 | 203288.57 | 64.17 | (59.04, 69.74) |
| hr/ml) | | | | |

Non-transformed data
least squares mean

| | | | | |
|---|---|---|---|---|
| Cₘₐₓ (ng/ml) | 9891.03 | 14852.34 | 66.60 | (58.31, 74.88) |
| AUC₀₋ₜ (ng-hr/ml) | 121911.10 | 204849.66 | 59.51 | (53.5, 65.52) |
| AUC_{0-INF} (ng- | 139803.88 | 219505.75 | 63.69 | (57.62, 69.76) |
| hr/ml) | | | | |
| Tₘₐₓ | 4.16 | 6.60 | 63.04 | (52.82, 73.26) |
| kₑ | 0.0741 | 0.0846 | 87.55 | (74.69, 100.4) |
| t_{1/2} | 11.07 | 9.12 | 121.37 | (108.97, 133.77) |

TABLE 12
Formulation 2C, Fasting versus Formulation 2C, Non-fasting; N = 57

| | | | | |
|---|---|---|---|---|
| | | | | 90% Confidence |
| | Formulation 2C | Formulation 2C | | Interval (Lower Limit, |
| PK variable | Fasting | Non-fasting | % Ratio | Upper Limit) |

Ln-transformed data
Geometric Mean

| | | | | |
|---|---|---|---|---|
| Cₘₐₓ (ng/ml) | 9184.69 | 9749.23 | 94.21 | (85.57, 103.72) |
| AUC₀₋ₜ (ng-hr/ml) | 112740.66 | 114042.17 | 98.86 | (90.99, 107.41) |
| AUC_{0-INF} (ng-hr/ml) | 130448.36 | 127826.23 | 102.05 | (93.9, 110.91) |

Non-transformed data
least squares mean

| | | | | |
|---|---|---|---|---|
| Cₘₐₓ (ng/ml) | 9891.03 | 10672.40 | 92.68 | (81.14, 104.21) |
| AUC₀₋ₜ (ng-hr/ml) | 121911.10 | 126368.34 | 96.47 | (86.73, 106.22) |
| AUC_{0-INF} (ng-hr/ml) | 139803.88 | 140244.83 | 99.69 | (90.19, 109.18) |
| Tₘₐₓ | 4.16 | 2.91 | 142.91 | (119.74, 166.08) |
| kₑ | 0.0741 | 0.0812 | 91.23 | (77.83, 104.62) |
| t_{1/2} | 11.07 | 10.51 | 105.37 | (94.6, 116.13) |

As shown by the results in Table 12, Formulation 2C administered in the fasting state is bioequivalent to administration in the non-fasting state, thus exhibiting no food effect.

### Example 7

### Relative Bioavailability Under Non-Fasting Conditions of the Extended-Release Coated Tablet Formulation 2D of Example 2 (60% Wax 6% Coat) Versus UROXATRAL® (10 mg); Relative Bioavailability of the Extended-Release Coated Tablet Formulation 2D of Example 2 Under Fasting Conditions Versus UROXATRAL® (10 mg) Under Non-Fasting Conditions; and Food Effect Evaluation with Formulation 2D of Example 2, Fasting Versus Non-Fasting

The study outlined in Example 6 is repeated with the extended-release coated tablet formulation 2D of Example 2 (60% wax 6% coat) and the results are provided in Tables 13-15 below.

TABLE 13
Formulation 2D, Non-Fasting versus UROXATRAL ®, Non-fasting; N = 55

| | | | | |
|---|---|---|---|---|
| | | | | 90% Confidence |
| | | | | Interval |
| | | Reference | % | (Lower Limit, |
| PK variable | Formulation 2D | (UROXATRAL ®) | Ratio | Upper Limit) |

Ln-transformed data
Geometric Mean

| | | | | |
|---|---|---|---|---|
| Cₘₐₓ (ng/ml) | 10370.40 | 14469.62 | 71.67 | (64.69, 79.41) |
| AUC₀₋ₜ (ng-hr/ml) | 111152.40 | 199427.13 | 55.74 | (51.14, 60.74) |
| AUC_{0-INF} (ng- | 128112.25 | 215900.21 | 59.34 | (54.89, 64.14) |
| hr/ml) | | | | |

Non-transformed data
least squares mean

| | | | | |
|---|---|---|---|---|
| Cₘₐₓ (ng/ml) | 11147.41 | 15660.50 | 71.18 | (62.48, 79.88) |
| AUC₀₋ₜ (ng- | 120450.06 | 216724.60 | 55.58 | (49.54, 61.62) |
| hr/ml) | | | | |
| AUC_{0-INF} (ng- | 137278.43 | 232117.55 | 59.14 | (53.56, 64.72) |
| hr/ml) | | | | |
| Tₘₐₓ | 3.09 | 6.45 | 47.92 | (36.67, 59.16) |
| kₑ | 0.0727 | 0.0804 | 90.44 | (82.23, 98.65) |
| t_{1/2} | 11.10 | 9.31 | 119.26 | (107.78, 130.73) |

TABLE 14
Formulation 2D, Fasting versus UROXATRAL ®, Non-fasting; N = 55

| | | | | |
|---|---|---|---|---|
| | | | | 90% |
| | | | | Confidence Interval |
| | Formulation | Reference | % | (Lower Limit, |
| PK variable | 2D | (UROXATRAL ®) | Ratio | Upper Limit) |

Ln-transformed data
Geometric Mean

| | | | | |
|---|---|---|---|---|
| Cₘₐₓ (ng/ml) | 8211.50 | 14469.62 | 56.75 | (51.22, 62.88) |
| AUC₀₋ₜ (ng-hr/ml) | 109370.40 | 199427.13 | 54.84 | (50.32, 59.77) |
| AUC_{0-INF} (ng-hr/ml) | 129411.42 | 215900.21 | 59.94 | (55.45, 64.79) |

Non-transformed data
least squares mean

| | | | | |
|---|---|---|---|---|
| Cₘₐₓ (ng/ml) | 9028.36 | 15660.50 | 57.65 | (48.95, 66.35) |
| AUC₀₋ₜ (ng-hr/ml) | 120863.12 | 216724.60 | 55.77 | (49.73, 61.81) |
| AUC_{0-INF} (ng-hr/ml) | 140614.06 | 232117.55 | 60.58 | (55, 66.16) |
| Tₘₐₓ | 4.08 | 6.45 | 63.30 | (52.05, 74.55) |
| kₑ | 0.0671 | 0.0804 | 83.42 | (75.21, 91.62) |
| t_{1/2} | 12.23 | 9.31 | 131.31 | (119.84, 142.79) |

TABLE 15
Formulation 2D, Fasting versus Formulation 2D, Non-fasting; N = 55

| | | | | |
|---|---|---|---|---|
| | | | | 90% |
| | | | | Confidence |
| | | | | Interval |
| | Formulation 2D | Formulation 2D | | (Lower Limit, |
| PK variable | Fasting | Non-fasting | % Ratio | Upper Limit) |

Ln-transformed data
Geometric Mean

| | | | | |
|---|---|---|---|---|
| Cₘₐₓ (ng/ml) | 8211.50 | 10370.40 | 79.18 | (71.47, 87.73) |
| AUC₀₋ₜ (ng-hr/ml) | 109370.40 | 111152.40 | 98.40 | (90.28, 107.24) |
| AUC_{0-INF} (ng-hr/ml) | 129411.42 | 128112.25 | 101.01 | (93.45, 109.19) |

Non-transformed data
least squares mean

| | | | | |
|---|---|---|---|---|
| Cₘₐₓ (ng/ml) | 9028.36 | 11147.41 | 80.99 | (68.77, 93.21) |
| AUC₀₋ₜ (ng-hr/ml) | 120863.12 | 120450.06 | 100.34 | (89.48, 111.21) |
| AUC_{0-INF} (ng-hr/ml) | 140614.06 | 137278.43 | 102.43 | (93, 111.86) |
| Tₘₐₓ | 4.08 | 3.09 | 132.10 | (108.63, 155.57) |
| kₑ | 0.0671 | 0.0727 | 92.23 | (83.16, 101.31) |
| t_{1/2} | 12.23 | 11.10 | 110.11 | (100.48, 119.73) |

As shown by the results in Table 15, Formulation 2D administered in the fasting state is substantially bioequivalent to administration in the non-fasting state, thus exhibiting no food effect.

### Example 8

### Relative Bioavailability Under Non-Fasting Conditions of the Extended-Release Coated Tablet Formulation 2B of Example 2 (50% Wax 3% Coat) or Formulation 2F of Example 2 (50% Wax 6% Coat) Versus UROXATRAL® (10 mg); Relative Bioavailability of the Extended-Release Coated Tablet Formulation 2B or 2F of Example 2 Under Fasting Conditions Versus UROXATRAL® (10 mg) Under Non-Fasting Conditions; and Food Effect Evaluation with Formulation 2B or 2F of Example 2, Fasting Versus Non-Fasting

The study is an open-label, single dose, randomized, five-period, five-treatment, fed/fasted bioequivalence and food effect study of two different 10 mg alfuzosin HCl ER tablet formulations (formulation 2B of Example 2 (50% wax 3% coat) or formulation 2F of Example 2 (50% wax 6% coat) when administered under fed/fasted conditions as compared to reference listed drug alfuzosin HCl extended-release tablet (10 mg, UROXATRAL®, Sanofi-Aventis) when administered under fed conditions (high fat breakfast) to healthy adult male volunteers. A food-effect evaluation is also performed with both alfuzosin HCl ER 10 mg tablet formulations, 2B or 2F administered in a fasted state to the same group of healthy adult male volunteers. The study is performed on 65 subjects. Each subject receives a single 10 mg dose of alfuzosin HCl, in either a fasted or fed state, on five separate occasions in a randomly assigned sequence, separated by seven day washout periods. During each study period, all subjects receive either a single dose of formulation 2B or 2F under fasted conditions, a single dose of formulation 2B or 2F under fed conditions (high fat breakfast), or a single dose of UROXATRAL® under fed conditions depending upon their randomization scheme following a minimum overnight fast of at least ten hours. For the fed treatment arms, the dose will be administered within five minutes of completing a standard, high-fat breakfast, and the subjects will have thirty minutes in which to complete the entire breakfast. Blood samples are drawn from each subject for drug content analysis at time zero (predose) and after dose administration, at time 0.25, then every ½ hour for the first eight hours, then at hours 9, 10, 12, 16, 20, 24, 36, and 48. Alfuzosin plasma concentrations in the blood samples are measured using a validated bioanalytical method.

The pharmacokinetic parameters are calculated as previously described and the results are provided in Tables 16-21 below.

TABLE 16
Formulation 2B, Non-Fasting versus UROXATRAL ®, Non-fasting; N = 65

| | | | | |
|---|---|---|---|---|
| | | | | 90% |
| | | | | Confidence Interval |
| | Formulation | Reference | % | (Lower Limit, |
| PK variable | 2B | (UROXATRAL ®) | Ratio | Upper Limit) |

Ln-transformed data
Geometric Mean

| | | | | |
|---|---|---|---|---|
| Cₘₐₓ (ng/ml) | 18069.32 | 14547.61 | 124.21 | (113.54, 135.87) |
| AUC₀₋ₜ (ng-hr/ml) | 192669.99 | 191937.01 | 100.38 | (91.31, 110.35) |
| AUC_{0-INF} (ng-hr/ml) | 206150.64 | 207303.09 | 99.44 | (91.34, 108.27) |

Non-transformed data
least squares mean

| | | | | |
|---|---|---|---|---|
| Cₘₐₓ (ng/ml) | 19288.83 | 15870.27 | 121.54 | (112.08, 131) |
| AUC₀₋ₜ (ng-hr/ml) | 210232.23 | 214816.21 | 97.87 | (90.51, 105.22) |
| AUC_{0-INF} (ng-hr/ml) | 222867.54 | 228666.58 | 97.46 | (90.47, 104.46) |
| Tₘₐₓ | 4.49 | 6.58 | 68.33 | (58.54, 78.13) |
| kₑ | 0.0923 | 0.0918 | 100.50 | (92.29, 108.71) |
| t_{1/2} | 8.38 | 8.13 | 103.14 | (94.16, 112.12) |

TABLE 17
Formulation 2B, Fasting versus UROXATRAL ®, Non-fasting; N = 65

| | | | | |
|---|---|---|---|---|
| | | | | 90% |
| | | | | Confidence Interval |
| | Formulation | Reference | % | (Lower Limit, |
| PK variable | 2B | (UROXATRAL ®) | Ratio | Upper Limit) |

Ln-transformed data
Geometric Mean

| | | | | |
|---|---|---|---|---|
| Cₘₐₓ (ng/ml) | 12904.02 | 14547.61 | 88.70 | (81.09, 97.03) |
| AUC₀₋ₜ (ng-hr/ml) | 166717.03 | 191937.01 | 86.86 | (79.01, 95.49) |
| AUC_{0-INF} (ng-hr/ml) | 181724.65 | 207303.09 | 87.66 | (80.52, 95.44) |

Non-transformed data
least squares mean

| | | | | |
|---|---|---|---|---|
| Cₘₐₓ (ng/ml) | 19032.41 | 15870.27 | 119.92 | (110.46, 129.39) |
| AUC₀₋ₜ (ng-hr/ml) | 214891.60 | 214816.21 | 100.04 | (92.68, 107.39) |
| AUC_{0-INF} (ng-hr/ml) | 227903.61 | 228666.58 | 99.67 | (92.67, 106.66) |
| Tₘₐₓ | 4.51 | 6.58 | 68.50 | (58.71, 78.3) |
| kₑ | 0.0849 | 0.0918 | 92.49 | (84.27, 100.7) |
| t_{1/2} | 9.12 | 8.13 | 112.13 | (103.15, 121.11) |

TABLE 18
Formulation 2B, Fasting versus Formulation 2B, Non-fasting: N = 65

| | | | | |
|---|---|---|---|---|
| | | | | 90% |
| | | | | Confidence Interval |
| | Formulation 2B | Formulation 2B | | (Lower Limit, |
| PK variable | Fasting | Non-fasting | % Ratio | Upper Limit) |

Ln-transformed data
Geometric Mean

| | | | | |
|---|---|---|---|---|
| Cₘₐₓ (ng/ml) | 12904.02 | 18069.32 | 71.41 | (65.28, 78.12) |
| AUC₀₋ₜ (ng-hr/ml) | 166717.03 | 192669.99 | 86.53 | (78.71, 95.13) |
| AUC_{0-INF} (ng-hr/ml) | 181724.65 | 206150.64 | 88.15 | (81.03, 95.89) |

Non-transformed data
least squares mean

| | | | | |
|---|---|---|---|---|
| Cₘₐₓ (ng/ml) | 12754.60 | 15870.27 | 80.37 | (70.9, 89.83) |
| AUC₀₋ₜ (ng-hr/ml) | 170492.12 | 214816.21 | 79.37 | (72.02, 86.72) |
| AUC_{0-INF} (ng-hr/ml) | 184260.25 | 228666.58 | 80.58 | (73.55, 87.62) |
| Tₘₐₓ | 4.45 | 6.58 | 67.71 | (57.91, 77.5) |
| kₑ | 0.0834 | 0.0918 | 90.84 | (82.58, 99.09) |
| t_{1/2} | 9.72 | 8.13 | 119.56 | (110.53, 128.58) |

TABLE 19
Formulation 2F, Non-Fasting versus UROXATRAL ®, Non-fasting; N = 65

| | | | | |
|---|---|---|---|---|
| | | | | 90% |
| | | | | Confidence Interval |
| | Formulation | Reference | % | (Lower Limit, |
| PK variable | 2F | (UROXATRAL ®) | Ratio | Upper Limit) |

Ln-transformed data
Geometric Mean

| | | | | |
|---|---|---|---|---|
| Cₘₐₓ (ng/ml) | 17376.93 | 14547.61 | 119.45 | (109.19, 130.67) |
| AUC₀₋ₜ (ng-hr/ml) | 192978.20 | 191937.01 | 100.54 | (91.46, 110.53) |
| AUC_{0-INF} (ng-hr/ml) | 206894.62 | 207303.09 | 99.80 | (91.67, 108.66) |

Non-transformed data
least squares mean

| | | | | |
|---|---|---|---|---|
| Cₘₐₓ (ng/ml) | 19032.41 | 15870.27 | 119.92 | (110.46, 129.39) |
| AUC₀₋ₜ (ng-hr/ml) | 214891.60 | 214816.21 | 100.04 | (92.68, 107.39) |
| AUC_{0-INF} (ng-hr/ml) | 227903.61 | 228666.58 | 99.67 | (92.67, 106.66) |
| Tₘₐₓ | 4.51 | 6.58 | 68.50 | (58.71, 78.3) |
| kₑ | 0.0849 | 0.0918 | 92.49 | (84.27, 100.7) |
| t_{1/2} | 9.12 | 8.13 | 112.13 | (103.15, 121.11) |

TABLE 20
Formulation 2F, Fasting versus UROXATRAL ®, Non-fasting; N = 65

| | | | | |
|---|---|---|---|---|
| | | | | 90% |
| | | | | Confidence Interval |
| | Formulation | Reference | % | (Lower Limit, |
| PK variable | 2F | (UROXATRAL ®) | Ratio | Upper Limit) |

Ln-transformed data
Geometric Mean

| | | | | |
|---|---|---|---|---|
| Cₘₐₓ (ng/ml) | 11580.06 | 14547.61 | 79.60 | (72.77, 87.08) |
| AUC₀₋ₜ (ng-hr/ml) | 150907.84 | 191937.01 | 78.62 | (71.52, 86.43) |
| AUC_{0-INF} (ng-hr/ml) | 166419.58 | 207303.09 | 80.28 | (73.7, 87.44) |

Non-transformed data
least squares mean

| | | | | |
|---|---|---|---|---|
| Cₘₐₓ (ng/ml) | 12754.60 | 15870.27 | 80.37 | (70.9, 89.83) |
| AUC₀₋ₜ (ng-hr/ml) | 170492.12 | 214816.21 | 79.37 | (72.02, 86.72) |
| AUC_{0-INF} (ng-hr/ml) | 184260.25 | 228666.58 | 80.58 | (73.55, 87.62) |
| Tₘₐₓ | 4.45 | 6.58 | 67.71 | (57.91, 77.5) |
| kₑ | 0.0834 | 0.0918 | 90.84 | (82.58, 99.09) |
| t_{1/2} | 9.72 | 8.13 | 119.56 | (110.53, 128.58) |

TABLE 21
Formulation 2F, Fasting versus Formulation 2F, Non-fasting; N = 65

| | | | | |
|---|---|---|---|---|
| | | | | 90% |
| | | | | Confidence Interval |
| | Formulation 2F | Formulation 2F | | (Lower Limit, |
| PK variable | Fasting | Non-fasting | % Ratio | Upper Limit) |

Ln-transformed data
Geometric Mean

| | | | | |
|---|---|---|---|---|
| Cₘₐₓ (ng/ml) | 11580.06 | 17376.93 | 66.64 | (60.92, 72.9) |
| AUC₀₋ₜ (ng-hr/ml) | 150907.84 | 192978.20 | 78.20 | (71.13, 85.97) |
| AUC_{0-INF} (ng-hr/ml) | 166419.58 | 206894.62 | 80.44 | (73.91, 87.54) |

Non-transformed data
least squares mean

| | | | | |
|---|---|---|---|---|
| Cₘₐₓ (ng/ml) | 12754.60 | 15870.27 | 80.37 | (70.9, 89.83) |
| AUC₀₋ₜ (ng-hr/ml) | 170492.12 | 214816.21 | 79.37 | (72.02, 86.72) |
| AUC_{0-INF} (ng-hr/ml) | 184260.25 | 228666.58 | 80.58 | (73.55, 87.62) |
| Tₘₐₓ | 4.45 | 6.58 | 67.71 | (57.91, 77.5) |
| kₑ | 0.0834 | 0.0918 | 90.84 | (82.58, 99.09) |
| t_{1/2} | 9.72 | 8.13 | 119.56 | (110.53, 128.58) |

As shown by the results in Tables 16-17 and 19-20, Formulations 2B and 2F administered in either the fasting or non-fasting state are substantially bioequivalent to UROXATRAL® administered in the non-fasting state.

Furthermore, Formulations 2B and 2F exhibit a reduced food affect (Tables 18 and 21.

### Example 9

### Relative Bioavailability Under Non-Fasting Conditions of the Extended-Release Coated Tablet Formulation 2A of Example 2 (45% Wax 3% Coat) or Formulation 2E of Example 2 (45% Wax 6% Coat) Versus UROXATRAL® (10 mg); Relative Bioavailability of the Extended-Release Coated Tablet Formulation 2A or 2E of Example 2 Under Fasting Conditions Versus UROXATRAL® (10 mg) Under Non-Fasting Conditions; and Food Effect Evaluation with Formulation 2A or 2E of Example 2, Fasting Versus Non-Fasting

The study outlined in Example 8 is repeated with the extended-release coated tablet formulations 2A (45% wax 3% coat) and 2E (45% wax 6% coat) of Example 2 and the results are provided in Tables 22-27 below.

TABLE 22
Formulation 2A, Non-Fasting versus UROXATRAL ®, Non-fasting; N = 65

| | | | | |
|---|---|---|---|---|
| | | | | 90% |
| | | | | Confidence Interval |
| | Formulation | Reference | % | (Lower Limit, |
| PK variable | 2A | (UROXATRAL ®) | Ratio | Upper Limit) |

Ln-transformed data
Geometric Mean

| | | | | |
|---|---|---|---|---|
| Cₘₐₓ (ng/ml) | 22367.83 | 14203.58 | 157.48 | (144.39, 171.76) |
| AUC₀₋ₜ (ng-hr/ml) | 209229.47 | 185303.76 | 112.91 | (105.37, 121) |
| AUC_{0-INF} (ng-hr/ml) | 221505.62 | 199731.78 | 110.90 | (103.19, 119.19) |

Non-transformed data
least squares mean

| | | | | |
|---|---|---|---|---|
| Cₘₐₓ (ng/ml) | 24642.56 | 15670.17 | 157.26 | (146.41, 168.1) |
| AUC₀₋ₜ (ng-hr/ml) | 228329.96 | 206112.99 | 110.78 | (104.34, 117.22) |
| AUC_{0-INF} (ng-hr/ml) | 240220.13 | 219056.90 | 109.66 | (101.52, 117.8) |
| Tₘₐₓ | 4.38 | 6.45 | 68.01 | (59.46, 76.56) |
| kₑ | 0.1043 | 0.0908 | 114.91 | (106.97, 122.84) |
| t_{1/2} | 7.58 | 8.64 | 87.80 | (0, 202.25) |

TABLE 23
Formulation 2A, Fasting versus UROXATRAL ®, Non-fasting; N = 65

| | | | | |
|---|---|---|---|---|
| | | | | 90% |
| | | | | Confidence Interval |
| | Formulation | Reference | | (Lower Limit, |
| PK variable | 2A | (UROXATRAL ®) | % Ratio | Upper Limit) |

Ln-transformed data
Geometric Mean

| | | | | |
|---|---|---|---|---|
| Cₘₐₓ (ng/ml) | 15460.79 | 14203.58 | 108.85 | (99.76, 118.77) |
| AUC₀₋ₜ (ng-hr/ml) | 174657.79 | 185303.76 | 94.25 | (87.93, 101.04) |
| AUC_{0-INF} (ng-hr/ml) | 188385.58 | 199731.78 | 94.32 | (87.73, 101.4) |

Non-transformed data
least squares mean

| | | | | |
|---|---|---|---|---|
| Cₘₐₓ (ng/ml) | 16861.00 | 15670.17 | 107.60 | (96.7, 118.5) |
| AUC₀₋ₜ (ng-hr/ml) | 188467.42 | 206112.99 | 91.44 | (84.97, 97.91) |
| AUC_{0-INF} (ng-hr/ml) | 202225.60 | 219056.90 | 92.32 | (84.13, 100.5) |
| Tₘₐₓ | 4.99 | 6.45 | 77.39 | (68.8, 85.98) |
| kₑ | 0.0943 | 0.0908 | 103.82 | (95.85, 111.8) |
| t_{1/2} | 8.44 | 8.64 | 97.75 | (0, 212.77) |

TABLE 24
Formulation 2A, Fasting versus Formulation 2A, Non-fasting; N = 65

| | | | | |
|---|---|---|---|---|
| | | | | 90% Confidence |
| | | | | Interval |
| | Formulation 2A | Formulation 2A | | (Lower Limit, |
| PK variable | Fasting | Non-fasting | % Ratio | Upper Limit) |

Ln-transformed data
Geometric Mean

| | | | | |
|---|---|---|---|---|
| Cₘₐₓ (ng/ml) | 15460.79 | 22367.83 | 69.12 | (63.35, 75.42) |
| AUC₀₋ₜ (ng-hr/ml) | 174657.79 | 209229.47 | 83.48 | (77.87, 89.49) |
| AUC_{0-INF} (ng-hr/ml) | 188385.58 | 221505.62 | 85.05 | (79.11, 91.43) |

Non-transformed data
least squares mean

| | | | | |
|---|---|---|---|---|
| Cₘₐₓ (ng/ml) | 16861.00 | 24642.56 | 68.42 | (61.49, 75.35) |
| AUC₀₋ₜ (ng-hr/ml) | 188467.42 | 228329.96 | 82.54 | (76.7, 88.38) |
| AUC_{0-INF} (ng-hr/ml) | 202225.60 | 240220.13 | 84.18 | (76.72, 91.65) |
| Tₘₐₓ | 4.99 | 4.38 | 113.79 | (101.16, 126.42) |
| kₑ | 0.0943 | 0.1043 | 90.36 | (83.42, 97.29) |
| t_{1/2} | 8.44 | 7.58 | 111.34 | (0, 242.34) |

TABLE 25
Formulation 2E, Non-Fasting versus UROXATRAL ®, Non-fasting; N = 65

| | | | | |
|---|---|---|---|---|
| | | | | 90% |
| | | | | Confidence Interval |
| | Formulation | Reference | % | (Lower Limit, |
| PK variable | 2E | (UROXATRAL ®) | Ratio | Upper Limit) |

Ln-transformed data
Geometric Mean

| | | | | |
|---|---|---|---|---|
| Cₘₐₓ (ng/ml) | 20036.65 | 14203.58 | 141.07 | (129.34, 153.86) |
| AUC₀₋ₜ (ng-hr/ml) | 202689.60 | 185303.76 | 109.38 | (102.07, 117.22) |
| AUC_{0-INF} (ng-hr/ml) | 214847.03 | 199731.78 | 107.57 | (100.09, 115.6) |

Non-transformed data
least squares mean

| | | | | |
|---|---|---|---|---|
| Cₘₐₓ (ng/ml) | 21473.62 | 15670.17 | 137.04 | (126.19, 147.88) |
| AUC₀₋ₜ (ng-hr/ml) | 216796.41 | 206112.99 | 105.18 | (98.75, 111.62) |
| AUC_{0-INF} (ng-hr/ml) | 228474.50 | 219056.90 | 104.30 | (96.16, 112.44) |
| Tₘₐₓ | 4.64 | 6.45 | 71.91 | (63.37, 80.46) |
| kₑ | 0.0984 | 0.0908 | 108.37 | (100.44, 116.3) |
| t_{1/2} | 8.13 | 8.64 | 94.11 | (0, 208.56) |

TABLE 26
Formulation 2E, Fasting versus UROXATRAL ®, Non-fasting; N = 65

| | | | | |
|---|---|---|---|---|
| | | | | 90% |
| | | | | Confidence Interval |
| | Formulation | Reference | % | (Lower Limit, |
| PK variable | 2E | (UROXATRAL ®) | Ratio | Upper Limit) |

Ln-transformed data
Geometric Mean

| | | | | |
|---|---|---|---|---|
| Cₘₐₓ (ng/ml) | 13221.85 | 14203.58 | 93.09 | (85.35, 101.53) |
| AUC₀₋ₜ (ng-hr/ml) | 164009.38 | 185303.76 | 88.51 | (82.59, 94.85) |
| AUC_{0-INF} (ng-hr/ml) | 183240.30 | 199731.78 | 91.74 | (85.37, 98.6) |

Non-transformed data
least squares mean

| | | | | |
|---|---|---|---|---|
| Cₘₐₓ (ng/ml) | 14302.33 | 15670.17 | 91.27 | (80.43, 102.12) |
| AUC₀₋ₜ (ng-hr/ml) | 178457.99 | 206112.99 | 86.58 | (80.14, 93.02) |
| AUC_{0-INF} (ng-hr/ml) | 202747.55 | 219056.90 | 92.55 | (84.41, 100.7) |
| Tₘₐₓ | 5.15 | 6.45 | 79.91 | (71.36, 88.46) |
| kₑ | 0.0894 | 0.0908 | 98.41 | (90.48, 106.34) |
| t_{1/2} | 18.25 | 8.64 | 211.24 | (96.79, 325.69) |

TABLE 27
Formulation 2E, Fasting versus Formulation 2E, Non-fasting; N = 65

| | | | | |
|---|---|---|---|---|
| | | | | 90% |
| | | | | Confidence Interval |
| | Formulation 2E | Formulation 2E | | (Lower Limit, |
| PK variable | Fasting | Non-fasting | % Ratio | Upper Limit) |

Ln-transformed data
Geometric Mean

| | | | | |
|---|---|---|---|---|
| Cₘₐₓ (ng/ml) | 13221.85 | 20036.65 | 65.99 | (60.5, 71.97) |
| AUC₀₋ₜ (ng-hr/ml) | 164009.38 | 202689.60 | 80.92 | (75.51, 86.71) |
| AUC_{0-INF} (ng-hr/ml) | 183240.30 | 214847.03 | 85.29 | (79.36, 91.66) |

Non-transformed data
least squares mean

| | | | | |
|---|---|---|---|---|
| Cₘₐₓ (ng/ml) | 14302.33 | 21473.62 | 66.60 | (58.69, 74.52) |
| AUC₀₋ₜ (ng-hr/ml) | 178457.99 | 216796.41 | 82.32 | (76.2, 88.44) |
| AUC_{0-INF} (ng-hr/ml) | 202747.55 | 228474.50 | 88.74 | (80.93, 96.55) |
| Tₘₐₓ | 5.15 | 4.64 | 111.12 | (99.23, 123) |
| kₑ | 0.0894 | 0.0984 | 90.81 | (83.49, 98.13) |
| t_{1/2} | 18.25 | 8.13 | 224.46 | (102.84, 346.07) |

As shown by the results in Tables 23 and 26, Formulations 2A and 2E administered in the fasting state are bioequivalent to UROXATRAL® administered in the non-fasting state.

Furthermore, Formulations 2A and 2E exhibit a reduced food affect (Tables 24 and 27.

A summary of the ln-transformed data, geometric mean are provided in Table 28 below.

TABLE 28
Summary of Ln-transformed data, geometric mean

| | | PK variable | | |
|---|---|---|---|---|
| | % Wax, | 90% Confidence Interval | | |
| | % coating | (Lower Limit, Upper Limit) | | |
| Formulations | wt gain | Cₘₐₓ | AUC₀₋ₜ | AUC_{0-INF} |
| 2A fasting:2A nonfasting | 45% wax | (63.35, 75.42) | (77.87, 89.49) | (79.11, 91.43) |
| | 3% coat | | | |
| 2B fasting:2B nonfasting | 50% wax | (65.28, 78.12) | (78.71, 95.13) | (81.03, 95.89) |
| | 3% coat | | | |
| 2C fasting:2C nonfasting | 60% wax | (85.57, 103.72) | (90.99, 107.41) | (93.9, 110.91) |
| | 4% coat | | | |
| 2D fasting:2D nonfasting | 60% wax | (71.47, 87.73) | (90.28, 107.24) | (93.45, 109.19) |
| | 6% coat | | | |
| 2E fasting:2E nonfasting | 45% wax | (60.5, 71.97) | (75.51, 86.71) | (79.36, 91.66) |
| | 6% coat | | | |
| 2F fasting:2F nonfasting | 50% wax | (60.92, 72.9) | (71.13, 85.97) | (73.91, 87.54) |
| | 6% coat | | | |
| 2G fasting:2G nonfasting | 60% wax | (70.84, 88.42) | (80.78, 96.16) | (80.69, 95.5) |
| | 12% coat | | | |
| 2H fasting:2H nonfasting | 60% wax | (70.5, 89.22) | (77.88, 96.9) | (78.57, 100.26) |
| | 20% coat | | | |
| 2A nonfasting: | 45% wax | (144.39, 171.76) | (105.37, 121) 10 | (103.19, 119.19) |
| UROXATRAL ® nonfasting | 3% coat | | | |
| 2B nonfasting: | 50% wax | (113.54, 135.87) | (91.31, 110.35) | (91.34, 108.27) |
| UROXATRAL ® nonfasting | 3% coat | | | |
| 2C nonfasting: | 60% wax | (64.5, 78.17) | (55.76, 65.83) | (57.86, 68.34) |
| UROXATRAL ® nonfasting | 4% coat | | | |
| 2D nonfasting: | 60% wax | (64.69, 79.41) | (51.14, 60.74) | (54.89, 64.14) |
| UROXATRAL ® nonfasting | 6% coat | | | |
| 2E nonfasting: | 45% wax | (129.34, 153.86) | (102.07, 117.22) | (100.09, 115.6) |
| UROXATRAL ® nonfasting | 6% coat | | | |
| 2F nonfasting: | 50% wax | (109.19, 130.67) | (91.46, 110.53) | (91.67, 108.66) |
| UROXATRAL ® nonfasting | 6% coat | | | |
| 2G nonfasting: | 60% wax | (73.4, 91.61) | (67.41, 80.25) | (70.65, 83.62) |
| UROXATRAL ® nonfasting | 12% coat | | | |
| 2H nonfasting: | 60% wax | (42.98, 54.4) | (41.81, 52.02) | (50.77, 64.78) |
| UROXATRAL ® nonfasting | 20% coat | | | |
| 2A fasting:UROXATRAL ® | 45% wax | (99.76, 118.77) | (87.93, 101.04) | (87.73, 101.4) |
| nonfasting | 3% coat | | | |
| 2B fasting:UROXATRAL ® | 50% wax | (81.09, 97.03) | (79.01, 95.49) | (80.52, 95.44) |
| nonfasting | 3% coat | | | |
| 2C fasting:UROXATRAL ® | 60% wax | (60.76, 73.64) | (55.13, 65.08) | (59.04, 69.74) |
| nonfasting | 4% coat | | | |
| 2D fasting:UROXATRAL ® | 60% wax | (51.22, 62.88) | (50.32, 59.77) | (55.45, 64.79) |
| nonfasting | 6% coat | | | |
| 2E fasting:UROXATRAL ® | 45% wax | (85.35, 101.53) | (82.59, 94.85) | (85.37, 98.6) |
| nonfasting | 6% coat | | | |
| 2F fasting:UROXATRAL ® | 50% wax | (72.77, 87.08) | (71.52, 86.43) | (73.7, 87.44) |
| nonfasting | 6% coat | | | |

All embodiments using the “comprising” language can also be written using “consisting essentially of” or “consisting of” transitional phrases.

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An oral controlled-release formulation, comprising:
a core comprising
alfuzosin or a pharmaceutically acceptable salt, solvate, hydrate, crystalline form, or non-crystalline form thereof, and
a wax excipient, present at about 40 wt. % to about 55 wt. % based on the total weight of the core;
a release-retarding material, present at about 0.1 wt. % to about 10 wt. % based on the total weight of the core; and
an extended-release coating substantially surrounding the core comprising a release-retarding coating material, wherein the coating is present at about 2.0 wt. % to about 15 wt. % based on the total weight of the core.

2. The formulation of claim 1, wherein the wax excipient is carnauba wax, vegetable wax, fruit wax, microcrystalline wax, bees wax, hydrocarbon wax, paraffin wax, cetyl esters wax, non-ionic emulsifying wax, anionic emulsifying wax, candelilla wax, stearyl alcohol, cetyl alcohol, cetostearyl alcohol, lauryl alcohol, myristyl alcohol, a hydrogenated vegetable oil, a hydrogenated castor oil, a fatty acid, a fatty acid ester, a fatty acid glyceride, a polyethylene glycol having a Mₙ, of greater than about 3000, or a combination comprising at least one of the foregoing wax excipients.

3. The formulation of claim 1, wherein the wax excipient is carnauba wax.

4-7. (canceled)

8. The formulation of claim 1, wherein the release-retarding material is an acrylic polymer, an alkylcellulose, a substituted alkylcellulose, shellac, zein, polyvinylpyrrolidine, crosslinked polyvinylpyrrolidinone, a vinyl acetate copolymer, a polyethylene oxide, a polyvinyl alcohol, or a combination comprising at least one of the foregoing materials.

9. The formulation of claim 1, wherein the release-retarding material is ethyl cellulose.

10. (canceled)

11. The formulation of claim 1, wherein the release-retarding coating material comprises a film forming polymer, wherein the film forming polymer is an alkylcellulose, a carboxyalkylcellulose, an alkali metal salt of a carboxyalkylcellulose, a carboxyalkyl alkylcellulose, a carboxyalkylcellulose ester, a starch, a pectin, a chitine derivate, a polysaccharide, a carrageenan, a galactomannas, traganth, agar-agar, gum arabicum, guar gum, xanthan gum, a polyacrylic acid, a polymethacrylic acid, a methacrylate copolymer, a polyvinylalcohol, a copolymer of polyvinylpyrrolidone with vinyl acetate, a polyalkylene oxide, a copolymer of ethylene oxide and propylene oxide, or a combination comprising at least one of the foregoing film forming polymers.

12. The formulation of claim 11, wherein the film forming polymer is ethylcellulose, carboxymethyl cellulose, sodium carboxymethylcellulose, carboxymethyl ethylcellulose, carboxymethylcellulose ester, sodium carboxymethylamylopectine, chitosan, alginic acid, alkali metal salt of alginic acid, ammonium salt of alginic acid, or a combination comprising at least one of the foregoing film forming polymers.

13. The formulation of claim 11, wherein the film forming polymer is ethylcellulose.

14. The formulation of claim 1, wherein the extended-release coating further comprises a pore former, wherein the pore former is a hydrophilic polymer, a hydroxy alkyl-alkyl cellulose, a hydroxyl alkyl cellulose, a povidone, a saccharide, an inorganic salt, a sugar alcohol, a polyoxyethylene sorbitan fatty acid ester, a hydrophilic methyacrylate copolymer, or a combination comprising at least one of the foregoing pore formers.

15. The formulation of claim 14, wherein the pore former is a hydroxypropylmethyl cellulose.

16. (canceled)

17. The formulation of claim 1, wherein the extended-release coating is present at about 2.5 to about 12 wt. % based on the total weight of the core and extended-release coating.

18. The formulation of claim 1, wherein the extended-release coating is present at about 3.5 to about 7 wt. % based on the total weight of the core and extended-release coating.

19. (canceled)

20. The formulation of claim 1, wherein the formulation administered as 10 mg dosage strength exhibits a 90% confidence interval for a ratio of a geometric mean of logarithmic transformed AUC_{0-∞} of the formulation administered in a fasted state to a geometric mean of logarithmic transformed AUC_{0-∞} of reference drug New Drug Application No. 021287, (10 milligrams) administered in a nonfasted state of about 0.80 to about 1.25 when tested in a group of five or more healthy humans.

21. (canceled)

22. The formulation of claim 1, wherein the formulation administered as 10 mg dosage strength exhibits a 90% confidence interval for a ratio of a geometric mean of logarithmic transformed Cₘₐₓ of the formulation administered in a fasted state to a geometric mean of logarithmic transformed Cₘₐₓ of reference drug New Drug Application No. 021287, (10 milligrams) administered in a non-fasted state of about 0.70 to about 1.43 when tested in a group of five or more healthy humans.

23. The formulation of claim 1, wherein the formulation administered as 10 mg dosage strength exhibits a 90% confidence interval for a ratio of a geometric mean of logarithmic transformed Cₘₐₓ of the formulation to a geometric mean of logarithmic transformed Cₘₐₓ of reference drug New Drug Application No. 021287, (10 milligrams)) of about 0.80 to about 1.25 when tested in a group of five or more healthy humans in the non-fasting state.

24. The formulation of claim 1, wherein the formulation exhibits a 90% confidence interval for a ratio of a geometric mean of logarithmic transformed AUC_{0-∞} of the formulation administered in a non-fasted state to a geometric mean of logarithmic transformed AUC_{0-∞} of the formulation administered in a fasted state of about 0.80 to about 1.25 when tested in a group of five or more healthy humans.

25. The formulation of claim 1, wherein the formulation exhibits a 90% confidence interval for a ratio of a geometric mean of logarithmic transformed 1 AUC₀₋ₜ of the formulation administered in a non-fasted state to a geometric mean of logarithmic transformed AUC₀₋ₜ of the formulation administered in a fasted state of about 0.80 to about 1.25 when tested in a group of five or more healthy humans.

26-27. (canceled)

28. The formulation of claim 1, wherein the formulation exhibits a reduced food effect such that a maximum alfuzosin plasma concentration (Cₘₐₓ) of the formulation administered in a fasted state is no more than 25% lower than when the formulation is administered in a non-fasted state when tested in a group of five or more healthy humans.

29-35. (canceled)

36. An oral controlled-release formulation, comprising:
a core comprising
alfuzosin hydrochloride, and
about 40 wt. % to about 55 wt. % carnauba wax based on the total weight of the core;
about 0.1 wt. % to about 10 wt. % ethyl cellulose based on the total weight of the core and
about 2.0 wt. % to about 15 wt. % based on the total weight of the core of an extended-release coating substantially surrounding the core, the extended-release coating comprising ethyl cellulose and hydroxypropyl methylcellulose.

37. A method of treating a patient, comprising
administering the formulation of claim 1 to a patient.

38. (canceled)

39. A method of reducing food effects associated with administration of alfuzosin, comprising
administering to a patient in need of treatment for benign prostatic hyperplasia, urinary retention, urinary problems associated with multiple sclerosis, chronic prostatitis, lower urinary tract symptoms, female sexual dysfunction, premature ejaculation, primary dysmenorrheal, managing blood pressure, or delaying onset of male ejaculation, an oral controlled release alfuzosin dosage form,
wherein the oral controlled release dosage form comprises
a core comprising about 1 wt. % to about 20 wt. % alfuzosin or a pharmaceutically acceptable salt, solvate, hydrate, crystalline form, or non-crystalline form thereof;
a wax excipient, present at about 40 wt. % to about 70 wt. % based on the total weight of the core; and
a release-retarding material present at about 1 wt. % to about 10 wt. % based on the total weight of the core; and
an extended-release coating substantially surrounding the core, present at about 1.0 wt. % to about 25 wt. % based on the total weight of the core and the extended-release coating.
